(19) United States
(12) Patent Application Publication
(10) Pub. No.: US 2010/0037330 A1
(43) Pub. Date: Feb. 11, 2010

(54) Efficient Somatic Cell Nuclear Transfer In Fish

(76) Inventors: Kannika Siripattarapravat, Lansing, MI (US); Jose B. Cibelli, East Lansing, MI (US)

Correspondence Address: HUNTON & WILLIAMS LLP;INTELLECTUAL PROPERTY DEPARTMENT, 1900 K STREET, N.W., SUITE 1200, WASHINGTON, DC, 20006-1109, US

(21) Appl. No.: 12/482,878

(22) Filed: Jun. 11, 2009

Related U.S. Application Data
(60) Provisional application No. 61/060,660, filed on Jun. 11, 2008.

(51) Int. Cl. A01K 67/027; C12N 13/00; C12N 15/873; C12N 5/00; C12N 15/01

(52) U.S. Cl. 800/20; 435/173.1; 435/455; 435/325; 435/446

(57) ABSTRACT

The present disclosure provides methods of producing enucleated cells by photoablation. Such enucleated cells may be used as recipient cells for Somatic Cell Nuclear Transfer and cloning. The nuclear donor and/or enucleated recipient cells may be any fish cells, such as zebrafish, koi, or medaka fish cells. Such methods may be used to efficiently produce transgenic fish including by way of example zebrafish, koi, and medaka fish.

### CROSS-REFERENCE TO RELATED APPLICATIONS

This application claims the benefit of U.S. Provisional Application Ser. No. 61/060,660, filed Jun. 11, 2008, which is hereby incorporated by reference in its entirety.

### BACKGROUND

1. Field of the Art

This disclosure generally relates to the field of therapeutic cloning and somatic cell nuclear transfer. Specifically, method are provided for producing enucleated cells by photoablation of recipient cell DNA. In another aspect, the disclosure relates to uses of such enucleated cells, such as Somatic Cell Nuclear Transfer (SCNT) and cloning. In a preferred embodiment the nuclear donor and/or enucleated recipient cells in SCNT are mammalian or fish cells, such as zebrafish cells.

In another aspect, the present disclosure describes a method for removal of DNA from a fish egg without compromising its developmental capacity, and methods for SCNT using such enucleated oocytes, as well as the use of such methods to efficiently produce transgenic zebrafish.

2. Description of Related Art

Zebrafish represents a vertebrate that serves as a convenient, relatively inexpensive, and useful model for the study of normal and pathological animal development, physiology, aging, cell death, and disease. Desirable characteristics possessed by zebrafish include its fecundity, external fertilization, rapid embryonic development, and a short generation interval (1,2). Zebrafish eggs are large and transparent, allowing for DNA injection, cell labeling, and transplantation (3). Similar to worms and flies, large scale mutagenesis and screening in zebrafish have proven to work effciently (4,5). However, these ‘forward genetic’ approaches are highly laborious and time-consuming (6).

A reliable and simple ‘reverse genetics’ method is desirable to bring the zebrafish model system into parity with more common rodent model systems. Knock-out/in mice are routinely generated using gene targeting homologous recombination in embryonic stem (ES) cells and subsequently production of germline-transmitted chimeras. Fan et al. have shown that germline competent ES cells can be obtained from zebrafish and later the same group showed that gene targeting can be done with these ES cells (7,8). However, as of yet, there are no reports on the generation of germline-competent founder animals using the aforementioned approach.

Cloned organisms may be produced by Somatic Cell Nuclear Transfer (SCNT), in which somatic cell nuclei are transplanted into enucleated oocytes. Such cells may give rise to embryos or animals genetically identical to the donor cell. When the donor nucleus is derived from a transgenic cell, transgenic embryos and animals may be produced.

Somatic cell nuclear transfer (SCNT) has the potential to become the method of choice for germline genetic modification in fish (9). However, conventional SCNT methods are technically demanding, requiring removal of the chorion (shell) of the egg and mechanical enucleation, e.g. using a glass needle. Eggs without the chorion start the process of division and therefore the efficiency of somatic cell nuclear transfer is compromised. Furthermore, such methods transfers the new nucleus anywhere in the animal pole of the egg, which may further decrease efficiency. The first and only published description of cloned zebrafish reported that nuclear transfer with cultured cells is possible, however, the efficiency of cloned fish production over the total number of eggs manipulated has remained at 2% or less (3, 21, 22).

Since the first reportedly successful SCNT experiments seven years ago (3), we and others have attempted multiple times to replicate the published protocol to no avail. Besides the innate low efficiency of the cloning procedure itself, multiple factors may have hindered the reproducibility of the method described, including 1) the use of activated eggs as a recipient which limits manipulation time to less than 1 hour after egg collection; 2) the technical challenge of blindly removing the egg's chromosomes; 3) the manipulation of dechorinated eggs, and 4) handling of the fragile reconstructed embryos. Zebrafish eggs undergo parthenogenetic activation easily. If spawned eggs come in contact with a hypotonic solution—even for a few seconds—they exit metaphase, chorion detaches, and a clear cytoplasmic stream toward the animal pole begins to form. For reasons not yet determined, however, the pronase treatment for removal of chorion, as performed by Lee, et al (2002) (3), triggers egg activation (15). No holding media has been found to inhibit such spontaneous activation (15). Moreover, naked eggs are easily broken by the suction of an egg holder or sharp-point injection needles. Furthermore, the egg membrane breaks as soon as it is exposed to either air or mineral oil.

### SUMMARY

This disclosure provides reliable and reproducible methodology for nuclear transfer in fish. For example, healthy and fertile clones have been generated from zebrafish strains AB, Tuebingen and AB/Tuebingen F1 (see Examples below). Using this technique, and depending on the strain of fish used, 1 to 13% of cloned hatch fry can be obtained from donor cells derived from adult fin and embryonic tail clip, respectively. A complete matched identity between donor cell and cloned fish produced by this technique was demonstrated by both phenotypic and genotypic analysis. The golden phenotype or GFP of transgenic Tuebingen—long fin, as presented in cloned animals, as well as SNP analysis, confirmed that no genetic trait of the recipient egg was carried over to cloned fish produced by this technique. The exposure of control eggs to both Hoechst DNA staining and UV irradiation showed no detrimental effect to embryonic development following in vitro fertilization. Cloned fish have a normal karyotype and produced offspring that carry their genetic traits. These methods can be readily used or modified for use in other zebrafish strains and in other fish species.

In an exemplary embodiment, non-activated recipient eggs can be enucleated by targeted energy, and donor nuclei can be introduced. For example, the recipient nucleus can be visualized by DNA staining of the metaphase plate, and can be ablated within the chorion using a laser. The laser-targeted ablation technique can completely inactivate the genome of the egg. The donor nucleus can be transferred into a recipient egg through the micropyle, a sperm entry site. The resulting constructed embryos can then be activated and allowed to develop. For example, constructed embryos can be activated in egg water and allowed to develop.

In exemplary embodiments, the Somatic Cell Nuclear Transfer (SCNT) methods employ mature, arrested eggs at MII as recipients, with complete inactivation of the egg's genome using a laser firing device which leaves the egg's chorion intact; delivery of the donor cells through the micropyle using a micropipette that breaks the cell membrane just prior to injection; activation of the reconstructed embryos in egg water; and development of cloned embryos with their chorion intact allowing for real-time monitoring. A clone's genetic identity to its parent can be confirmed by methods known in the art, including phenotypic screening, genotyping (e.g., using single nucleotide polymorphism (SNP) markers), and karyotyping (e.g. by replication banding) (see, for example, reference 10).

In one aspect, the disclosure relates to a method for making an enucleated cell comprising: visualizing the nucleus of a cell; and irradiating the nucleus of the cell with a laser; whereby the nuclear DNA of the cell is ablated. The enucleated cell may an oocyte, such as a fish oocyte, such as a Zebrafish oocyte. Visualizing the cell nucleus may comprise contacting the cell with a nuclear stain, such as a Hoechst dye, such as Hoechst 33342. Visualizing the cell nucleus may comprise observing expression of a fluorescent protein in the cell nucleus. The fluorescent protein may comprise a green, blue, yellow, or cyan fluorescent protein; and/or may comprise a fusion protein, such as a histone. The method may further comprise transplanting a donor nucleus into the enucleated cell, such as through the micropyle of an enucleated egg. The donor nucleus may be is transgenic. The donor nucleus may be of the same species as the enucleated egg, of the same genus as the enucleated egg, and/or of a different species than the enucleated egg. The donor nucleus may be mammalian, such as human.

In another aspect, the disclosure relates to a method for making an enucleated cell comprising: visualizing the nucleus of a cell; and irradiating the nucleus of the cell with a laser; whereby the nucleus of the cell is ablated. The method may further comprise transplanting a donor nucleus into the enucleated cell.

In another aspect, the disclosure relates to a method for making an enucleated cell comprising: contacting a cell with a nuclear stain; and irradiating the cell with a laser; whereby the nucleus of the cell is ablated. The method may further comprise transplanting a donor nucleus into the enucleated cell.

In another aspect, the disclosure relates to an enucleated cell produced by the foregoing methods.

In another aspect, the disclosure relates to a nuclear transplant cell produced by the foregoing methods.

In another aspect, the disclosure relates to an organism produced by the foregoing methods.

### BRIEF DESCRIPTION OF THE DRAWINGS

FIG. 1 illustrates a protocol for SCNT according to an exemplary embodiment of the present disclosure.

FIG. 2 illustrates recipient eggs before and after nuclear photoablation.

FIG. 3 illustrates the homozygous golden phenotype of cloned zebrafish and their offspring.

FIG. 4 illustrates abnormalities observed in some cloned embryos.

FIG. 5 illustrates karyotyping and genotyping of cloned fish.

FIG. 6 illustrates the number of eggs of various genotypes to reach different developmental stages.

FIG. 7 shows primers used for genotyping of cloned fish.

### DETAILED DESCRIPTION

Conventional methods of producing enucleated cells require removal of the chorion (shell) of the egg. When the chorion is removed the zebrafish eggs start division prematurely, thus efficiency is reduced.

In contrast, the present disclosure provides energetic methods for ablation of the recipient cell nuclear DNA, such that the chorion may be left intact. Specifically, the nuclear DNA may be ablated with energy, preferably focused or targeted energy, more preferably laser light, more preferably infrared laser light. The energy may be focused on the cell, for example through the objective of an inverted microscope. In a preferred embodiment, the energy may be focused on the cell nuclear DNA. In another embodiment, a nuclear stain may preferentially absorb the energy, such that the nuclear DNA is specifically ablated whether or not the energy is focused on the cell nucleus. For example, enucleation may be performed by contacting cells with a nuclear stain or a nucleus-targeted fluorophore and irradiating the cells with light that excites or is highly absorbed by the stain or fluorophore, whereby the energy is preferentially absorbed in the nucleus and the nucleus is preferentially ablated. In another embodiment, cells may be irradiated with energy that is preferentially absorbed by or particularly destructive to the nucleus or to DNA, for example x-rays, gamma rays, or ultraviolet rays. High-throughput enucleation may be performed by irradiating whole cells with energy that is preferentially absorbed by or particularly destructive to the nucleus or to DNA (optionally potentiated by a nuclear stain or nuclear-targeted fluorophore). The amount of energy to which whole cells are exposed can be controlled by means known in the art, such as by passing cells through a stream to control duration and intensity of exposure to the energy source (e.g., using a flow cytometer); suspending cells in liquid in a container of defined geometry (such as a cuvette) and passing a controlled amount of radiation through the container; arranging cells in a thin or single layer (for example in a culture dish) and exposing to an energy source; etc. Enucleated cells produced by high-throughput methods can then be fused or injected with donor nuclei, e.g., manually or using high-throughput methods such as induced fusion with donor cells.

Without intent to be limited by theory, it is believed that the methods described herein overcomes the limitations of prior SCNT methodology (3) due to the retention of the chorion. The intact chorion is believed to provide a natural protection for the eggs and cloned embryos, making eggs more tolerant to micromanipulation and injection and facilitating monitoring of developing cloned embryos.

Laser-assisted ablation of the metaphase plate of the egg showed complete inactivation of its genomic DNA. Fluorescence DNA staining, enabled visualization of the metaphase plate and direct laser-ablation of the egg genome. Although the use of Hoechst 33342 and exposure of UV light have shown to be toxic to oocytes of some species, zebrafish eggs seem to tolerate both of them well, as in vitro fertilized embryos developed normally. Our results clearly demonstrated that the genetic material of recipient eggs was inactivated as cloned embryos showed golden phenotypes and DNA fingerprinting of the clones completely matched that of the donor cells. In addition, clones had normal karyotypes, were fertile at reproductive maturity age, and produced offspring that carried their genetic traits.

We have demonstrated that cloned fish embryos can be derived from donor cells of both embryonic and adult origins, with greater developmental potency generally observed for embryo-derived donor cells. On average, at least one cloned fish per person per day is obtained using embryo-derived donor cells. The identity of cloned fish can easily be verified using initial phenotypic screening and later genotyping using SNP markers described.

In certain embodiments, the cell or cell nucleus may be visualized, such that the energy to be targeted to the cell or cell nucleus. Such visualization includes visualization by a technician, such as a human, as well as automated visualization, for example using a camera coupled to computer, which can then target the energy for nuclear ablation. The cell and/or cell nucleus may be visualized using methods known in the art, such as nuclear stains, visible light, light refraction microscopy, the LC-POLSCOPE system (see http://www.cri-inc.com/files/LCPS_IM_%20Brochure.pdf, visited Jun. 11, 2008), CRI OOSIGHT™ Imaging System (see http://www.criinc.com/files/CRi %20 SCNT %20Full %20Protocol %20Final.pdf, visited Jun. 11, 2008), and expression of a fluorescent protein (such as GFP, YFP, BFP, CFP and derivatives thereof) which may be optionally targeted to the nucleus, e.g. expressed as histone fusion proteins. In a one embodiment, the cell nucleus may be visualized using a nuclear stain, introduced into the cell through diffusion or microinjection. Any suitable nuclear stain may be used. In a preferred embodiment the nuclear stain may be Hoechst 33258 or Hoechst 33342.

In exemplary embodiments, the cell is contacted with a nuclear stain, and then the nuclear DNA is ‘burned’ or ‘ablated’ with the laser. The chorion may be left intact, such that the egg does not start division prematurely. The method transfers the nucleus or chromosomal DNA of the donor cell or the donor cell itself via the micropyle, a funnel-shaped hole in the chorion.

In exemplary embodiments, the organism from which the recipient cell is derived is of the same species as the nuclear donor. Alternatively, the organism from which the recipient cell is derived is of a different species than the nuclear donor (see, e.g. Zhu and Sun et al., Cell Research (2000), 10, 17-27). For example, a cell of mammalian origin, including human, may be used as the nuclear donor, and the recipient cell may be of another species, including zebrafish and other fish, whereby cells of a different lineage than the nuclear donor are generated (transdifferentiation occurs).

In an exemplary embodiment, the fish develops into an adult. Optionally the adult fish are fertile. In a preferred embodiments, the transgenic fish are euploid, i.e. have a diploid number of chromosomes. Alternatively the transgenic fish are aneuploid (e.g. have a triploid or tetraploid number of chromosomes).

In exemplary embodiments, the source of donor and/or recipient cells may be a Zebrafish strain, which include: AB (AB), AB/C32 (AB/C32), AB/TL (AB/TL), AB/Tuebingen (AB/TU), C32 (C32), Cologne (KOLN), Darjeeling (DAR), Ekkwill (EKW), HK/AB (HK/AB), HK/Sing (HK/SING), Hong Kong (HK), India (IND), Indonesia (INDO), Nadia (NA), RIKEN WT (RW), Singapore (SING), SJA (SJA), SJD (SJD), SJD/C32 (SJD/C32), Tuebingen (TU), Tupfel long fin (TL), Tupfel long fin nacre (TLN), WIK (WIK), WIK/AB (WIK/AB), and other hybrids of these strains in addition to those specifically recited. The donor and recipient strains may be the same or different.

In exemplary embodiments, the source of donor and/or recipient cells may be a fish of the Danio genus, which include Danio albolineatus, Danio abolineatus var pulcher, Danio abolineatus var tweediei, Danio choprae, Danio dangila, Daniofeegradei, Danio kerri, Danio kyathit var spotted, Danio kyathit var striped, Danio meghalayensis, Danio nigrofasciatus, Danio roseus, Danio rerio, Danio rerio var frankei, Danio sp “Hikari”, Danio sp aff kyathit, Danio sp “KP01”, Danio sp “TW01”, Danio sp “TW02”, Danio sp “TW03”, and any hybrid between these species.

In exemplary embodiments, the source of donor and/or recipient cells may be a fish of the Devario genus, which include Devario acrostomus, Devario acuticephala, Devario aequippinatus, Devario affinis, Devario annandalei, Devario apogon, Devario apopyris, Devario assamensis, Devario browni, Devario chrysotaeniatus, Devario devario, Devario fangfangae, Devariofraseri, Devario gibber, Devario horai, Devario interruptus, Devario kakhienensis, Devario laoensis, Devario leptos, Devario manipurensis, Devario maetaengensis, Devario malabaricus, Devario naganensis, Devario neilgherriensis, Devario pathirana, Devario peninsulae, Devario quangbinhensis, Devario regina, Devario salmonata, Devario shanensis, Devario sondhii, Devario spinosus, Devario strigillifer, Devario suvatti, Devario yuensis, Devario sp. “Broken Line”, Devario sp. “giraffe”, and any hybrid between these species.

In exemplary embodiments, the source of donor and/or recipient cells may be a fish of the Rasbora genus, which include Rasbora borapetensis, Rasbora caverii, Rasbora daniconius, Rasbora dies, Rasbora dorsiocellata, Rasbora einthovenii, Rasbora elegans, Rasbora notura, Rasbora pauciperforata, Rasbora paviei, Rasbora rasbora, Rasbora trilineata, Rasbora vaterifloris, Rasbora wilpita, and any hybrid between these species.

In exemplary embodiments, the source of donor and/or recipient cells may be a fish of the Trigonostigma genus, which include Trigonostigma espei, Trigonostigma hengeli, Trigonostigma heteromorpha, Trigonostigma somphongsi, and any hybrid between these species.

In exemplary embodiments, the source of donor and/or recipient cells may be a fish of the Boraras genus, which include Boraras brigittae, Boraras maculatus, Boraras merah, Boraras micros, Boraras uropthalmoides, and any hybrid between these species.

In exemplary embodiments, the source of donor and/or recipient cells may be a fish of the Tanichthys genus, which include Tanichthys albonubes, Tanichthys micagemmae, and any hybrid between these species.

In exemplary embodiments, the source of donor and/or recipient cells may be a fish of the Pseudorasbora genus, which include Pseudorasbora elongata, Pseudorasbora parva, Pseudorasbora cf. parva, Pseudorasbora pumila, Pseudorasbora pumila pumila, Pseudorasbora sp. SPH-2006, and any hybrid between these species.

In exemplary embodiments, the source of donor and/or recipient cells may be a fish of the Cyprinus genus, which include Cyprinus acutidorsalis, Cyprinus barbatus, Cyprinus carpio, Cyprinus daliensis, Cyprinus ilishaestomus, Cyprinus intha, Cyprinus longipectoralis, Cyprinus mahuensis, Cyprinus megalophthalmus, Cyprinus micristiusk, Cyprinus multitaeniata, Cyprinus pellegrini, Cyprinus robrofuscus, Cyprinus yilongensis, Cyprinus yunnanensis, and any hybrid between these species. For example, the donor and/or recipient cells may be koi. For example, even from carefully chosen high-quality or champion-grade parents, the majority of koi offspring are often of inferior quality (e.g., lacking desired coloration) or genetically defective. Cloning of koi by the methods described herein may decrease variability in offspring and improve the likelihood of obtaining desired attributes. Recipient cells may be koi eggs, which can be obtained from a high-quality or regular-quality female, and can be harvested from naturally laid eggs, by “stripping” or “squeezing” (a manual procedure known in the art for removing eggs), by dissection, or other means known in the art. Donor cells can be derived from cultured cells, biopsies or other tissue samples (including whole organisms), etc. Exemplary named koi varieties include Gosanke, Kōhaku, TaishōSanshoku, Shōwa Sanshoku, Tanchō, Asagi, Utsurimono, Bekko, Goshiki, Shūsui, Kinginrin, Kawarimono, Ōgon, Kumonryū, Doitsu-goi, Ochiba, Koromo, Hikari-moyomono, Ghost koi, Butterfly koi, though numerous other varieties (which may or may not be particularly named) may also be employed in accord with these methods.

In exemplary embodiments, the source of donor and/or recipient cells may be any organism that exhibits a low efficiency of establishing clones or cell lines by SCNT methods employing mechanical enucleation. These methods may be employed, for example, with any species or source of recipient cells that have low efficiency of establishing clones or cell lines due to the use of activated eggs as a recipient which limits manipulation time after egg collection; due to the technical challenge of blindly removing the recipient cell's chromosomes; due to the fragility of recipient cells; due to fragility of dechorinated eggs; due to fragility of reconstructed embryos; due to undesired parthenogenetic activation; and/or due to unknown or uncertain causes. For example, source of donor and/or recipient cells may be a fish or other organisms in which recipient cells undergo parthenogenetic activation or apoptosis due to preparation for mechanical enucleation (e.g., removal of a chorion if present) and/or due to mechanical enucleation itself. As another example, the source of donor and/or recipient cells may be a fish or other organisms in which it is difficult to identify the suitable site for implantation of the nucleus during or after mechanical enucleation. For example, these methods may be employed in fish species in which the preferred site of nuclear implantation is the animal pole but removal of the chorion and mechanical enucleation makes the animal pole more difficult to locate. As another example, the source of donor and/or recipient cells may be a fish or other organisms in which the recipient cells are fragile or become fragile during SCNT.

In exemplary embodiments, the source of donor and/or recipient cells may be a hybrid between fish of different species or genera, e.g. different species within the Danio genus and the Devario genus or a cross between species of those genera. For example, a desired hybrid fish may be difficult to obtain due to low viability and/or phenotypic variation, but once the desired hybrid is obtained it may be propagated by the methods described herein.

Optionally the source of the donor and/or recipient cell is infertile, for example, a sterile hybrid.

The following references which describe general zebrafish laboratory methods are hereby incorporated by reference in their entireties: Westerfield, M. (2000). The zebrafish book. A guide for the laboratory use of zebrafish (Danio rerio). 4th ed., Univ. of Oregon Press, Eugene, and Westerfield, M. (2007) THE ZEBRAFISH BOOK, 5th Edition; A guide for the laboratory use of zebrafish (Danio rerio), Eugene, University of Oregon Press.

In exemplary embodiments, the source of donor and/or recipient cells may be any of the following fish: Abalistes stellatus, Abramites hypselonotus, Abudefduf saxatilis, Acanthemblemaria spinosa, Acanthochromis polyacanthus, Acanthurus achilles, Acanthurus chirurgus, Acanthurus coeruleus, Acanthurus dussumieri, Acanthurus japonicus, Acanthurus leucosternon, Acanthurus lineatus, Acanthurus maculiceps, Acanthurus nigricans, Acanthurus nigrofuscus, Acanthurus nigroris, Acanthurus olivaceus, Acanthurus pyroferus, Acanthurus sohal, Acanthurus tennenti, Acanthurus triostegus, Acanthurus tristis, Acanthurus xanthopterus, Acantopsis choirorhynchus, Acantopsis octoactinotos, acaras, Acarichthys heckelii, Achilles tang, Adolfo's catfish, Aequidens pulcher, Aequidens rivulatus, Afra cichlid, African butterfly cichlid, African cichlids, African glass catfish, African Grouper, African lungfish, African moony, Agamyxis pectinifrons, Agassiz's dwarf cichlid, aholehole, airbreathing catfish, airsac catfish, Alaska blackfish, Albacore, Alectis indicus, Alewife, Alfonsino, Algae eater, Allauad's haplo, Allen's Dottyback, alligatorfish, Altum angelfish, Amago, Amazon sailfin catfish, Amblyapistus taenionotus, Amblycirrhitus pinos, Amblyeleotris diagonalis, Amblyeleotris guttata, Amblyeleotris randalli, Amblyeleotris steinitzi, Amblyeleotris wheeleri, Amblyglyphidodon aureus, Amblygobius decussatus, Amblygobius hectori, Amblygobius phalaena, Amblygobius rainfordi, Ambon Chromis, Ambon damsel, Ambon Scorpionfish, American cichlids, American Flagfish, American sole, Amphilophus citrinellus, Amphilophus labiatus, Amphiprion clarkii, Amphiprion frenatus, Amphiprion melanopus, Amphiprion ocellaris, Amphiprion percula, Amphiprion perideraion, Amphiprion polymnus, Amphiprion sebae, Amur pike, Anableps spp., Anchovy, Ancistrus spp., Anemonefish, Angel shark, Angelfish, Angelfish (Dwarf), Angelfish (Large), Anglemouth, Angler, Angler catfish, Anglerfish, Anisotremus virginicus, Annularis Angelfish, Anomalochromis thomasi, Antarctic cod, Antarctic dragonfish, Antarctic icefish, Antenna codlet, Antennarius commerson, Antennarius maculatus, Antennarius multiocellatus, Antennarius striatus, Antenneta Lionfish, Anthias, Aphyocharax anisitsi, Apistogramma agassizii, Apistogramma bitaeniata, Apistogramma borellii, Apistogramma cacatuoides, Apistogramma eremnopyge, Apistogramma nijsseni, Apistogramma trifasciata, apistogrammas, Apogon aureus, Apogon compressus, Apogon cyanosoma, Apogon flores, Apogon fragilis, Apogon gilberti, Apogon hoeveni, Apogon leptacanthus, Apogon maculatus, Apogon nigrofasciatus, Apolemichthys griffisi, Apolemichthys xanthurus, Apteronotus albifrons, Apteronotus leptorhynchus, Arabian dottyback, Arapaima, Arc eye hawkfish, Archamia zosterophora, Archerfish, Archocentrus nigrofasciatus, Archocentrus sajica, Arctic char, Aristichthys nobilis, Arius berneyi, Arius graeffei, Arius seemanni, Armored catfish, Armored gurnard, Armored searobin, Armorhead, Armorhead catfish, Arothron hispidus, Arothron immaculatus, Arothron manilensis, Arothron mappa, Arothron meleagris, Arothron nigropunctatus, Arothron stellatus, Arowana, Arrowtooth eel, Aruana, Arulius barb, Asfur Angelfish, Asian carps, Asian redtail catfish, Asiatic glassfish, Aspidoras fuscoguttatus, Aspidoras lakoi, Aspidoras pauciradiatus, Aspidoras rochai, Assasi trigger, Assessor flavissimus, Assessor macneilli, Assessors, Astatoreochromis alluaudi, Astatotilapia aenocolor, Astatotilapia elegans, Astatotilapia latifasciata, Astatotilapia nubila, Astatotilapia piceatus, Astatotilapia schubotziellus, Astatotilapia sp Red Tail, Astatotilapia sp. Spot Bar, Astronotus ocellatus, Astyanax mexicanus, Atelomycterus marmoratus, Atka mackerel, Atlantic blue tang, Atlantic cod, Atlantic eel, Atlantic herring, Atlantic Mudskipper, Atlantic salmon, Atlantic saury, Atlantic silverside, Atlantic spadefish, Atlantic trout, Atrosalarias fuscus, Aulonocara baenschi, Aulonocara jacobfreibergi, Aulonocara steveni, Auratus cichlid, Aurora, Australasian salmon, Australian grayling, Australian herring, Australian lungfish, Australian multicolor pseudochromis, Australian prowfish, Australian rainbowfish, Australian shark catfish, Ayu, Azure Damsel, Baikal oilfish, Bala shark, Balantiocheilus melanopterus, Balistapus undulatus, Balistes punctatus, Balistes vetula, Balistoides conspicillum, Balistoides viridescens, ballan wrasse, bamboo shark, Banana Wrasse, Banded archerfish, Banded corydoras, Banded Eel, banded killifish, Banded Pipefish, Banded Snake Eel, Banded sole, bandfish, bandit corydoras, Banggai Cardinal, bango, bangu, Banjo catfish, banjo catfish, Bannerfish, barb, barbel, barbeled dragonfish, barbeled houndshark, barbelless catfish, Barbonymus schwanenfeldii, Barbs, Barbus brevipinnis, Barbus callipterus, Barbus ticto, barfish, barracuda, barracudina, barramundi, Barred Angelfish, barred danio, Barred Hamlet, Barred Mudskipper, Barredtail corydoras, barreleye, Barrier Reef Chromis, Bartlett's anthias, Baryancistrus spp., basking shark, Bass, bass, basslet, Basslets, bat ray, Batfish, batfish, beachsalmon, beaked salmon, beaked sandfish, beardfish, Beaufortia kweichowensis, Bellus Angelfish, beluga sturgeon, Bengal danio, bengal danio, Bengal loach, bent-tooth, Benthochromis tricoti, Berney's shark catfish, betta, Betta splendens, bichir, Bichirs, Bicolor Angelfish, Bicolor Anthias, Bicolor Blenny, Bicolor Foxface, Bicolor Goatfish, Bicolor Parrotfish, Bicolor pseudochromis, Big Eye Soldierfish, bigeye, bigeye squaretail, bighead carp, bigmouth buffalo, bigscale, bigscale fish, bigscale pomfret, Bigspot barb, billfish, Biotodoma cupido, Bird Wrasse, bitterling, Black and Gold Chromis, Black and Gold damsel, black angelfish, Black banded cat shark, Black bar Chromis, Black Barred Convict goby, black bass, Black Cap Gramma, Black cap jawfish, Black clown goby, Black Combtooth Blenny, black dragonfish, Black Edge Moray Eel, Black ghost knifefish, Black Hamlet, black mackerel, Black molly, Black neon tetra, Black phantom tetra, black pickerel, black prickleback, Black Ribbon Eel, Black ruby barb, Black Sailfin Blenny, black scabbardfish, black scalyfin, black sea bass, Black Snapper, black swallower, Black tetra, black tetra, black triggerfish, Black-winged hatchetfish, Blackbar Soldierfish, blackchin, Blackchin tilapia, blackfish, Blackfoot Lionfish, Blackline Fang Blenny, blackline penguinfish, Blackline rasbora, Blackmargined damsel, blacksmelt, Blackstripe Cardinalfish, Blackstripe corydoras, Blacktip Grouper, blacktip reef shark, Blacktop corydoras, bleak, Bleeding heart tetra, Blennies, blenny, blind goby, blind shark, blind tetra, Blonde naso tang, Blood parrot cichlid, Blood Red hawkfish, blood-red jewel cichlid, Bloodfin tetra, Blue & Gold Blenny, Blue acara, Blue and gold damsel, Blue and Yellow Grouper, Blue Angelfish, Blue Assesor, Blue botia, blue catfish, Blue Chromis, Blue corydoras, Blue damsel, blue danio, Blue discus, Blue dolphin cichlid, Blue Dot Grouper, Blue dot jawfish, blue eye, Blue Eyed tang, Blue flavivertex pseudochromis, blue gourami, Blue Gudgeon Dartfish, Blue Hamlet, Blue jaw trigger/Blue throat trigger, Blue johanni cichlid, Blue Line Grouper, Blue line trigger, Blue Lined Surgeonfish, Blue ram, Blue Ribbon Eel, blue shark, blue triggerfish, Blue velvet damsel, blue whiting, Blue-headed tilefish, Blue-Lined Rabbitfish, blue-redstripe danio, Blueback damsel, Bluebarred Cardinalfish, Blueface Angelfish, Bluefin damsel, bluefin tuna, bluefish, bluegill, Bluehead Wrasse, Blueline demoiselle, Bluelined Dottyback, Bluespotted Angelfish, Bluespotted corydoras, Bluespotted Watchman goby, Bluestreak Cardinalfish, blunt-snout bream, bluntnose knifefish, bluntnose minnow, boafish, boarfish, bobtail snipe eel, bocaccio, Bodianus bilunulatus, Bodianus bimaculatus, Bodianus diana, Bodianus mesothorax, Bodianus pulchellus, Bodianus rufus, Boeseman's rainbowfish, boga, Bolivian ram, Bombay duck, bonefish, bonito, bonnetmouth, bonytail chub, bonylongue, Bothus lunatus, Botia almorhae, Botia dario, Botia kubotai, Botia macracantha, Botia striata, bottlenose, Boulengerochromis microlepsis, bowfin, Boxfish, boxfish, Brachydanio kerri, Brachydanio rerio, Brachygobius xanthozonus, bramble shark, Brazilian Flameback Angelfish, Brazilian Gramma, Brazilian Seahorse, bream, bristlemouth, bristlenose catfish, Bristlenose pleco, Bristletooth tang, Britski's catfish, broadband dogfish, Bronze corydoras, brook lamprey, brook trout, brotula, Brown Dottyback, Brown ghost knifefish, brown trout, Bucktooth tetra, Buenos Aires tetra, buffalofish, bull shark, bull trout, bullhead, bullhead shark, Bumblebee cichlid, Bumblebee goby, Bundoon Blenny, Bunocephalus coracoideus, burbot, buri, burma danio, Burmese border loach, burrowing goby, Bursa trigger, bushynose placo, Butter Hamlet, butterfish, butterfly peacock bass, butterfly ray, Butterflyfish, butterflyfish, Caerulean damsel, Caesio xanthonota, California flyingfish, California halibut, California smoothtongue, Callichthys callichthys, Calloplesiops altivelis, Cambodian log sucker, Canary Deep Water damsel, Canary Fang Blenny, canary rockfish, candiru, candlefish, Cantherhines dumerili, Canthigaster bennetti, Canthigaster coronata, Canthigaster jactator, Canthigaster papua, Canthigaster rostrata, Canthigaster valentini, capelin, Carassius auratus, Carassius auratus gibelio, Cardinal tetra, Cardinalfish, cardinalfish, Carinotetraodon travancoricus, Carnegiella marthae, Carnegiella strigata, carp, Carpenter's fairy wrasse, carpetshark, carpsucker, Cascarudo, cat shark, Catalina goby, catalufa, Catfish, catfish, catla, Cave tetra, Cave Transparent goby, cavefish, Celebes Rainbowfish, Celebes rainbowfish, central mudminnow, Centropyge acanthops, Centropyge argi, Centropyge aurantonotus, Centropyge bicolor, Centropyge bispinosa, Centropyge eibli, Centropyge ferrugata, Centropyge flavicauda, Centropyge flavissima, Centropyge heraldic Centropyge loricula, Centropyge multicolor, Centropyge multifasciata, Centropyge potteri, Centropyge tibicens, Centropyge vroliki, cepalin, Cephalopholis argus, Cephalopholis formosa, Cephalopholis fulva, Cephalopholis miniatus, Cephalopholis panamensis, Cephalopholis polleni, Cephalopholis spiloparaea, Cephalopholis taeniops, Cephalopholis urodelus, Cephalopholis urodeta, Cetoscarus bicolor, Chaetodermis penicilligerus, Chaetodon auriga, Chaetodon falcula, Chaetodon lunula, Chaetodon mertensii, Chaetodon paucifasciatus, Chaetodon rafflesii, Chaetodon semilarvatus, Chaetodon tinkeri, Chaetodon ulietensis, Chaetodon unimaculatus, Chaetodontoplus caeruleopunctatus, Chaetodontoplus duboulayi, Chaetodontoplus meridithii, chain pickerel, Chainlink Moray Eel, Chalinochromis, Chalk Bass, Champsochromis spilorhyncus, channel bass, channel catfish, Chao Phraya giant shark, char, characiformes, Characins, Checker barb, Checkerboard Cichlid, Cheeklined Maori Wrasse, Cheilinus diagrammus, Chelmon rostratus, Chemy barb, Chemy Dottyback, cherry salmon, Cherubfish, Chevron tang, Chiloscyllium plagiosum, Chiloscyllium punctatum, chimaera, Chinese algae eater, Chinese high fin banded shark, Chinese hillstream loach, Chinook salmon, Chipokee cichlid, Chocolate cichlid, Chocolate gourami, Chocolate tang, Choerodon fasciatus, Christmas Wrasse, Chromidotilapia guentheri, Chromis, Chromis amboinensis, Chromis atripectoralis, Chromis chromis, Chromis cyanea, Chromis flavomaculata, Chromis insolatus, Chromis iomelas, Chromis limbaughi, Chromis lineata, Chromis nitida, Chromis retrofasciata, Chromis viridis, Chromis xanthura, Chrysipetra caeruleolineata, Chrysipetra cyanea, Chrysipetra rex, Chrysipetra rollandi, Chrysiptera cyanea, Chrysiptera galba, Chrysiptera hemicyanea, Chrysiptera parasema, Chrysiptera springeri, Chrysiptera starcki, Chrysiptera talboti, Chrysiptera taupou, Chrysiptera tricincta, chub, chubsucker, chum salmon, Cichla orinocensis, Cichlasoma managuense, Cichlasoma meeki, Cichlasoma octofasciatum, Cichlasoma urophthalmus, cichlasomas, cichlid, Cichlids, Cinnamon Anemonefish, Cirrhilabrus aurantidorsalis, Cirrhilabrus cyanopleura, Cirrhilabrus exquisitus, Cirrhilabrus filamentosus, Cirrhilabrus flavidorsalis, Cirrhilabrus jordani, Cirrhilabrus laboutei, Cirrhilabrus lineatus, Cirrhilabrus lubbocki, Cirrhilabrus luteovittatus, Cirrhilabrus lyukyuensis, Cirrhilabrus punctatus, Cirrhilabrus rhomboidalis, Cirrhilabrus rubrisquamis, Cirrhilabrus rubriventralis, Cirrhilabrus scottorum, Cirrhilabrus solorensis, Cirrhitichthys aprinus, Cirrhitichthys aureus, Cirrhitichthys falco, Cirrhitichthys fasciatus, Cirrhitichthys oxycephalus, Cirrhitichthys polyactis, Cirripectes stigmaticus, cisco, Citron clown goby, Clarias batrachus, Clarkii Anemonefish, Cleithracara maronii, climbing catfish, climbing gourami, climbing perch, clingfish, Clipper barb, Cloudy damsel, Clown barb, Clown filefish, Clown loach, clown loach, clown sailfin pleco, Clown tang, Clown trigger, clown triggerfish, Clownfish, clownfish, Cobalt blue cichlid, cobalt zebra cichlid, cobbler, cobia, Cockatoo dwarf cichlid, cod, cod icefish, codlet, codling, coelacanth, coffinfish, coho salmon, Cold-water cyprinids, coley, Colisa lalia, collared carpetshark, collared dogfish, Colombian shark catfish, Colorado squawfish, Colored filefish, Colossoma bidens, combfish, combtail gourami, combtooth blenny, common barb, common carp, Common dace, Common discus, Common hatchetfish, Common pleco, Common syno, common tunny, Coney Grouper, conger eel, Congo pufferfish, Congo tetra, Congrogadus subducens, Convict Blenny, convict blenny, Convict cichlid, convict cichlid, Convict tang, cookie-cutter shark, coolie loach, Cooper's Anthias, Copadichromis borleyi, Copperbanded Butterflyfish, Coral beauty Angelfish, Coral cat shark, Coral hawkfish, Coral Hogfish, Coris formosa, Coris gaimard, cornetfish, Cortez Angelfish, Cortez Rainbow Wrasse, Corydoras acutus, Corydoras adolfoi, Corydoras aeneus, Corydoras ambiacus, Corydoras atropersonatus, Corydoras axelrodi, Corydoras bondi, Corydoras britskii, Corydoras caudimaculatus, Corydoras cochui, Corydoras ehrhardti, Corydoras elegans, Corydoras evelynae, Corydoras geoffroy, Corydoras guapore, Corydoras habrosus, Corydoras haraldschultzi, Corydoras hastatus, Corydoras latus, Corydoras leucomelas, Corydoras loxozonus, Corydoras macropterus, Corydoras melanistius, Corydoras melanotaenia, Corydoras metae, Corydoras multiradiatus, Corydoras nanus, Corydoras narcissus, Corydoras nattereri, Corydoras ornatus, Corydoras osteocarus, Corydoras paleatus, Corydoras panda, Corydoras pastazensis, Corydoras polystictus, Corydoras prionotos, Corydoras pygmaeus, Corydoras reticulatus, Corydoras schwartzi, Corydoras semiaquilus, Corydoras septentrionalis, Corydoras simulatus, Corydoras sodalis, Corydoras splendens, Corydoras sterbai, Corydoras sychri, Corydoras trilineatus, Corydoras undulatus, Corydoras xinguensis, Coryphopterus glaucofraenum, Corythoichthys haematopterus, Court Jester Goby, cow shark, cowfish, cownose ray, crappie, creek chub, Crescent Banded Grunt, crestfish, crevice kelpfish, croaker, Croaking gourami, crocodile icefish, crocodile shark, Cromileptes altivelis, Cross' damsel, Crosshatch trigger, Crossocheilus siamensis, Crossosalarias macrospilus, crucian carp, Cryptocentrus aurora, Cryptocentrus cinctus, Cryptocentrus leptocephalus, Cryptocentrus pavoninoides, Crystal eyed catfish, Ctenochaetus binotatus, Ctenochaetus hawaiiensis, Ctenochaetus striatus, Ctenochaetus strigosus, Ctenocheatus tominiensis, Ctenogobiops tangaroai, Cuban Hogfish, cuchia, Cuckoo squeaker, cuckoo wrasse, cupid cichlid, cusk-eel, cuskfish, cutlassfish, cutthroat eel, cutthroat trout, Cyathopharynx, Cynotilapia afra, Cypho Purpurascens, Cyphotilapia frontosa, Cyphotilapia gibberosa, Cyphotilapia sp. North, Cyprichromis, Cyprinidontiformes, Cyprinids, cypriniforms, Cyprinus carpio, Cyrtocara moorii, dab, dace, daggertooth pike conger, Damselfish, damselfish, Damsels, danio, Danio albolineatus, Danio devario, Danio nigrofasciatus, danionins, Danios, darter, Dartfish, dartfish, Dascyllus albisella, Dascyllus aruanus, Dascyllus auripinnis, Dascyllus carneus, Dascyllus flavicaudus, Dascyllus marginatus, Dascyllus melanurus, Dascyllus reticulatus, Dascyllus trimaculatus, Datnioides microlepis, dealfish, Death Valley pupfish, Decorated squeaker, Decoy Scorpionfish, deep sea anglerfish, deep sea bonefish, deep sea eel, deep sea smelt, deepwater cardinalfish, deepwater flathead, deepwater stingray, delta smelt, demoiselle, Dendrochirus barberi, Dendrochirus biocellatus, Dendrochirus brachypterus, Dendrochirus zebra, denticle herring, desert pupfish, Desjardini tang, Devario, Devario aequipinnatus, Devario malabaricus, Devario regina, Devil lionfish, devil ray, Diadem Anthias, diadema basslet, Diademichthys lineatus, Diagonal Bar Prawn Goby, Diamond Blenny, Diamond Watchman Goby, Dicrossus filamentosus, Dilectus Dottyback, Dimidiochromis compressiceps, Diodon holocanthus, Diodon hystrix, Dischistodus prosopotaenia, discus, discuses, diver: New Zealand sand diver or Long-finned sand diver, Doctorfish, Dogface Pufferfish, dogfish, Dogfish Orientalis, dogfish shark, dogteeth tetra, dogtooth cichild, Dojo loach, dojo loach, Dolly Varden trout, Domino damsel, dorab, dorado, dory, Doryrhamphus dactyliophorus, Doryrhamphus janssi, Doryrhamphus pessuliferus, dottyback, Dracula goby, Dragon goby, dragon goby, Dragon Moray Eel, Dragon Wrasse, dragonet, Dragonets, Dragonface Pipefish, dragonfish, driftfish, driftwood catfish, drum fish, Duboulayi's rainbowfish, duckbill, duckbill eel, duckbilled barracudina, Duncker's barb, Dusky Batfish, Dusky Dottyback, dusky grouper, Dusky jawfish, Dussumieri tang, dwarf barb, Dwarf cichlids, Dwarf corydoras, Dwarf flag cichlid, Dwarf gourami, dwarf gourami, Dwarf loach, dwarf loach, Dwarf pufferfish, Dwarf Seahorse, eagle ray, Eartheater cichlid, earthworm eel, Eastern rainbowfish, Echidna catenata, Echidna nebulosa, Echidna polyzona, Ecsenius bicolor, Ecsenius bimaculatus, Ecsenius gravieri, Ecsenius lineatus, Ecsenius midas, Ecsenius namiyei, Ecsenius stigmatura, Ectodus descampsii, eel, eel cod, eel-goby, eelblenny, eelpout, Eels, eeltail catfish, Eibli Angelfish, Eibli mimic tang, Eight line wrasse, Elacatinus oceanops, Elacatinus puncticulatus, elasmobranch, electric catfish, Electric eel, electric eel, electric knifefish, electric ray, electric stargazer, Electric yellow cichlid, Electrophorus electricus, Elegant corydoras, Eleotris picta, elephantfish, elephantnose fish, Elongate Dottyback, elver, Ember Blenny, Emblemaria pandionis, Emerald catfish, emperor, Emperor Angelfish, emperor angelfish, emperor bream, Emperor Snapper, Emperor tetra, Enchelycore pardalis, Enchelyurus flavipes, Endler's livebearer, Engineer goby, Epalzeorhynchos bicolor, Epalzeorhynchos frenatum, Epalzeorhynchos kalopterus, Epaulette Shark, Epinephelus fasciatus, Epinephelus flavocaeruleus, Epinephelus summana, Erpetoichthys calabaricus, Eschmeyer's Scorpionfish, escolar, Etroplus maculatus, Etroplus suratensis, eucla cod, eulachon, Eureka red peacock, European chub, European eel, European flounder, European minnow, European perch, Euxiphipops xanthometopon, Even-Spotted squeaker, Eviota pellucida, Exodon paradoxus, Exquisite fairy wrasse, fairy cichlid, Falco's hawkfish, false brotula, false cat shark, false corydoras, false moray, False network catfish, False Percula, false Siamese algae eater, False spotted catfish, false trevally, false upside down catfish, fangtooth, Fantail orange filefish, Farlowella spp., fathead minnow, fathead sculpin, featherback, Featherfin, featherfin knifefish, Featherfin squeaker, Fiddler stingray, fierasfer, FIG. 8 pufferfish, Fiji blue devil damsel, Filefish, filefish, finback cat shark, Fine-spotted fairy wrasse, fingerfish, fire bar danio, Fire eel, Fire Fish, firefish, Firemouth cichlid, Firetail Dottyback, fiveband barb, flabby whalefish, flagblenny, flagfin, flagfish, flagtail, Flame Angelfish, Flame hawkfish, Flame Wrasse, Flameback, Flamefish, Flasher Scorpionfish, flashlight fish, flat loach, Flatfish, flatfish, flathead, flathead catfish, flier, flounder, Flowerhorn cichlid, flying characin, Flying fox (fish), flying gurnard, flyingfish, footballfish, Forcipiger flavissimus, forehead brooder, Formosa Wrasse, Four line wrasse, Four stripe damsel, Four-eyed fish, four-eyed fish, Foxface, Fragile Cardinalfish, Freckled hawkfish, French Angelfish, french angelfish, Freshwater angelfish, freshwater eel, freshwater flyingfish, freshwater hatchetfish, freshwater herring, freshwater shark, Fridmani pseudochromis, frigate mackerel, frilled shark, Frogfish, frogfish, frogmouth catfish, Frontosa cichild, Frostfin Cardinalfish, Fu Man Chu Lionfish, Fuelleborn's cichlid/Blue mbuna, Fusco, fusilier fish, Fuzzy dwarf lionfish, galjoen fish, Ganges shark, Gar, gar, garden eel, garibaldi, Garibaldi damsel, Garnet tetra, garpike, Garra cambodgiensis, Gasteropelecus levis, Gasteropelecus sternicla, Genicanthus bellus, Genicanthus semifasciatus, Geophagus altifrons, German ram, ghost carp, ghost flathead, ghost knifefish, ghost pipefish, ghoul, Giant Anglerfish, Giant danio, giant danio, Giant freshwater pufferfish, Giant gourami, giant gourami, Giant pangasius, giant sea bass, giant wels, gianttail, gibberfish, Gila trout, Gilbert's Cardinalfish, Ginglymostoma cirratum, giraffe cichild, Girdled Cardinalfish, gizzard shad, Glass catfish, glass catfish, Glass Eye Squirrelfish, glass knifefish, glassfish, glibel carp, Glossolepis incisus, Glossolepis pseudoincisus, Glossolepis wanamensis, glowlight danio, Glowlight tetra, Gnathanodon speciosus, Goatfish, goatfish, Gobies, Gobiodon acicularis, Gobiodon atrangulatus, Gobiodon citrinus, Gobiodon okinawae, Gobioides broussonnetii, Gobiosoma multifasciatum, goblin shark, goby, Gold barb, Gold Neon Eviota goby, Gold nugget pleco, Gold Rim tang, Goldbar Wrasse, Golden barb, golden dojo, Golden Dwarf Eel, Golden dwarf sucker, Golden Grouper, Golden Hamlet, Golden hawkfish, Golden Heart trigger, golden loach, Golden moray eel, golden oto, Golden Pilotfish, Golden Puffer, golden shiner, Golden Stripe Soapfish, golden trout, Goldenback trigger, goldeye, Goldfish, goldfish, Goldsaddle Goatfish, goldspotted killifish, gombessa, Gomphosus varius, goosefish, gopher rockfish, gouramie, Gramma brasiliensis, Gramma loreto, Gramma melacara, Grammistes sexlineatus, grass carp, graveldiver, Gray Angelfish, Gray bichir, gray eel-catfish, gray mullet, gray reef shark, grayling, Great Seahorse, great white shark, Green banded goby, Green chromide, Green Chromis, Green Clown goby, green discus, Green gold catfish, Green lionfish, Green moray eel, Green neon tetra, Green Scat, Green spotted puffer, Green swordtail, green swordtail, Green terror, Green Wrasse, Greenback fairy wrasse, greeneye, greenling, Greenstreaked Eartheater, Greenstripe barb, grenadier, grideye, Griffis Angelfish, ground shark, grouper, Groupers, grunion, grunt, grunt sculpin, grunter, Grunts, Guapore corydoras, gudgeon, Guenther's Mouthbrooder, guitarfish, gulf menhaden, gulper, gulper eel, gunnel, Guppies, Guppy, guppy, gurnard, Gymnocorymbus ternetzi, Gymnomuraena zebra, Gymnothorax favagineus, Gymnothorax fimbriatus, Gymnothorax funebris, Gymnothorax kidako, Gymnothorax melatremus, Gymnothorax meleagris, Gymnothorax miliaris, Gymnothorax nudivomer, Gymnothorax picta, Gymnothorax saxicola, Gyrinocheilus aymonieri, haddock, hagfish, hairtail, hake, Half and Half Chromis, Half-black Angelfish, half-gill, Half-Moon Angelfish, halfbeak, halfmoon, Halfmoon Trigger, Halfmoon trigger, halibut, Halichoeres chloropterus, Halichoeres chrysus, Halichoeres hortulanus, Halichoeres melanurus, Halichoeres trispilus, Halichores iridis, halosaur, Hamlet, hamlet, hammerhead shark, Hammerjaw, handfish, Haplochromis obliquedens, hardhead catfish, harelip sucker, Harlequin Bass, Harlequin rasbora, Harlequin tusk, hatchetfish, Hawaiian black trigger, Hawaiian Blue Puffer, Hawaiian Dascyllus, Hawaiian Hogfish, Hawaiian Saddle Puffer, Hawaiian Spotted Puffer, Hawkfish, hawkfish, Hector's goby, Helmet Cowfish, Helostoma temminckii, Hemibagrus wyckii, Hemibagrus wyckioides, Hemichromis bimaculatus, Hemichromis lifalili, Hemigrammus erythrozonus, Hemigrammus pulcher, Hemigrammus rhodostomus, Hemiscyllium ocellatum, Heniochus diphreutes, Herald's Angelfish, Herichthys cyanoguttatus, Heros severus, herring, herring smelt, Herring/Sardine cichlid, Heterodontus portusjacksoni, Heteropriacanthus cruentatus, Hexanematichthys seemanni, Hi Fin Red Banded goby, hillstream loach, Hippo Point Salmon, Hippo tang, Hippocampus abdominalis, Hippocampus bargibanti, Hippocampus breviceps, Hippocampus comes, Hippocampus erectus, Hippocampus histrix, Hippocampus kuda, Hippocampus reidi, Hippocampus Skelloggi, Hippocampus whitei, Hippocampus zosterae, Histrio histrio, Hoeven's Wrasse, hog sucker, Hogfish, Hognosed brochis, hoki, Holacanthus bermudensis, Holacanthus ciliaris, Holacanthus passer, Honey head damsel, Hongi, Hoplolatilus marcosi, Hoplolatilus purpureus, Hoplolatilus starcki, Horabagrus brachysoma, horn shark, hornet cichlid, Horseface loach, horsefish, Horseshoe Filefish, houndshark, huchen, humuhumu-nukunuku-apua′a, hussar, Hypancistrus zebra, Hyphessobrycon anisitsi, Hyphessobrycon erythrostigma, Hyphessobrycon herbertaxelrodi, Hyphessobrycon megalopterus, Hyphessobrycon pulchripinnis, Hyphessobrycon serpae, Hypoplectrus gemma, Hypoplectrus gummigutta, Hypoplectrus guttavarius, Hypoplectrus indigo, Hypoplectrus nigricans, Hypoplectrus puella, Hypoplectrus unicolor, Hypostomus plecostomus, Hypostomus punctatus, Hypselecara temporalis, Hypsophrys nematopus, Hypsypops rubicunda, icefish, ide, ilisha, Immaculate Puffer, inanga, inconnu, Indian black trigger, Indian glassy fish, Indian mul, Indian Threadfin, Indigo Hamlet, Inimicus didactylus, Iodotropheus sprengerae, Iracundus signifer, Iriatherina werneri, Iridescent shark, jack, Jack Dempsey, Jack Dempsey cichlid, jackfish, Jacks, Jade Filefish, Jaguar cichlid, Jansen Saddle Wrasse, Janss' Pipefish, Japanese eel, Japanese Swallow Angelfish, Jarbua terapon, Jawfish, jawfish, jellynose fish, Jewel cichlid, Jewel damsel, jewel tetra, Jeweled Moray Eel, jewelfish, jewfish, Johanni cichlid, john dory, Jordanella floridae, Julidochromis dickfeldi, Julidochromis marlieri, Julidochromis ornatus, Julidochromis regani, Julidochromis transcriptus, Julie cichlid, Kafue pike, kahawai, Kaligono, kaluga, kanyu, kappy, kelp perch, kelpfish, Kenyi cichlid, Keyhole Angelfish, Keyhole cichlid, Kidako Moray Eel, King Angelfish, King demoiselle, king of herring, king-of-the-salmon, Kingfish, kirigoldfish, Kissing gourami, kissing gourami, knifefish, knifejaw, Knight goby, Koi, koi, kokanee, kokopu, Kole tang, Koran Angelfish, krib, Kribensis, Kryptopterus bicirrhis, Kuhli loach, kuhli loach, L-046, Labeotropheus fuelleborni, Labidochromis caeruleus, Labidochromis sp. Hongi, Labidochromis sp. Mbamba Bay, Labout's Fairy Wrasse, Labracinus cyclophthalmus, Labyrinth fish, labyrinth fish, Laced Moray, Lacey Scorpionfish, Lactoria cornuta, ladyfish, Laetacara curviceps, lagena, lake chub, Lake Kurumoi rainbowfish, Lake Malawi cichlids, Lake Malawi syno, Lake Tanganyika cichlids, lake trout, Lake Wanam rainbowfish, lake whitefish, lampfish, lamprey, Lamprologus ocellatus, lancetfish, lanternfish, large-eye bream, largemouth bass, largenose fish, Latticed Butterflyfish, lavender cichild, Lavender tang, Lawnmower Blenny, Leaf Fish, leaffish, Leaflip Grouper, leatherjacket, lefteye flounder, Lei trigger, Leiarius marmoratus, Leiarius pictus, Lemon cichlid, Lemon damsel, lemon shark, Lemon tetra, Lemonpeel Angelfish, lenok, leopard catfish, leopard danio, Leopard sailfin pleco, Leopard Wrasse, Lepisosteus oculatus, Lesser spiny eel, Leuciscus leuciscus, Lifalili jewel cichlid, lightfish, lighthousefish, Limbaughi Chromis, limia, Linear Blenny, Lineatus Fairy Wrasse, Lined Chromis, Lined Seahorse, lined sole, ling, ling cod, Lionfish, lionfish, Live-bearers, livebearer, Livingston's cichlid, lizardfish, loach, loach catfish, loach goby, loach minnow, Loaches, Lobochilotes labiatus, long-finned char, long-finned pike, long-whiskered catfish, longfin, Longfin Dottyback, longfin dragonfish, longfin escolar, Longfin Fairy Wrasse, Longfin gregory, longfin smelt, Longhorn Cowfish, longjaw mudsucker, Longlure Frogfish, longneck eel, Longnose Butterflyfish, longnose chimaera, longnose corydoras, longnose dace, Longnose hawkfish, longnose lancetfish, Longnose loach, longnose sucker, longnose whiptail catfish, lookdown catfish, loosejaw, Lopezi tang, Lost River sucker, louvar, loweye catfish, luderick, Lufundi, luminous hake, lumphead cichlid, lumpsucker, lungfish, Lutjanus sebae, lyretail, Lyretail Anthias, Lyretail cichlid, Lyretail Dottyback, Lyretail Grouper, Lyretail hawkfish, Lyretail Wrasse, Lythrypnus dalli, mackerel, mackerel shark, Macolor niger, Macrognathus aculeatus, Macrognathus siamensis, Macropharyngodon geoffroyi, Macropharyngodon meleagris, Macropodus opercularis, madtom, Magnificent foxface, mahi-mahi, mahseer, mail-cheeked fish, Maingano, Majestic Angelfish, mako shark, Malabar danio, Malacoctenus boehlkei, Malawi barracuda, Malawi eyebiter, Malawi golden cichlid, man-of-war fish, managuense cichlid, Mandarinfish, manefish, Manonichthys alleni, Manonichthys polynemus, Manta Ray, Manybar Goatfish, Map Puffer, Marble Wrasse, Marbled hatchetfish, Marbled Headstander, marblefish, Marginated damsel, Marine angelfish, Marine Beta, marine hatchetfish, marlin, Maroon Clownfish, Marosatherina ladigesi, Masked Butterflyfish, Masked corydoras, Masked Julie, Mastacembelus armatus, Mastacembelus erythrotaenia, masu salmon, Mayan cichlid, Maylandia callainos, Maylandia lombardoi, McCosker's Flasher wrasse, medaka, medusafish, Megalobrama amblycephala, megamouth shark, Meiacanthus bundoon, Meiacanthus grammistes, Meiacanthus nigrolineatus, Meiacanthus oualanensis, Melanochromis auratus, Melanochromis aurora, Melanochromis chipokae, Melanochromis cyaneorhabdos, Melanochromis joanjohnsonae, Melanochromis johannii, Melanotaenia affinis, Melanotaenia boesemani, Melanotaenia duboulayi, Melanotaenia fluviatilis, Melanotaenia parva, Melanotaenia praecox, Melanotaenia splendida splendida, Melichthys indicus, Melichthys niger, Melichthys vidua, Melon barb, menhaden, merluccid hake, Merten's Butterflyfish, Metriaclima estherae, Metynnis argenteus, Meuschenia hippocrepis, Mexican blind cavefish, Mexican golden trout, Microspathodon chrysurus, Midas Blenny, Midas cichild, midshipman, Mikrogeophagus altispinosa, Mikrogeophagus ramirezi, milkfish, Mimic Filefish, Mimic tang, Miniatus Grouper, minnow, Misgurnus anguillicaudatus, Modoc sucker, mojarra, mola, mola mola, mollies, molly, monkeyface prickleback, monkfish, Monodactylus argenteus, Monodactylus sebae, mooneye, moonfish, Moonlight gourami, Moorish idol, mora, moray eel, morid cod, morwong, Mosaic corydoras, Moses sole, mosquitofish, mosshead warbonnet, mouthbrooder, Mozambique Scorpionfish, Mozambique tilapia, mrigal, mud cat, mud catfish, mud minnow, mudfish, mudminnow, Mudskipper, mudskipper, mudsucker, mullet, Multicolor Angelfish, Multicolor Lubbock's Wrasse, Multicolor velvet wrasse, mummichog, Muraena lentiginosa, murray cod, muskellunge, mustache triggerfish, mustard eel, Mycteroperca rosacea, Myrichthys colubrinus, Myrichthys maculosus, Myripristis jacobus, Myripristis vittata, Mystery wrasse, Myxocyprinus asiaticus, naked-back knifefish, Nannacara adoketa, Nannostomus trifasciatus, nase, Naso lituratus, Naso Lopezi, Naso tang, Naso unicornis, Naso Vlamingi, needlefish, Nemateleotris decora, Nemateleotris magnifica, Nematobrycon palmeri, Neocirrhitus armatus, Neoglyphidodon crossi, Neoglyphidodon melas, Neoglyphidodon nigroris, Neolamprologus brichardi, Neolamprologus leleupi, Neolamprologus multifasciatus, Neolamprologus similis, Neon damsel, neon dottyback, Neon goby, Neon pseudochromis, Neon rainbowfish, Neon tetra, neon tetra, Neopomacentrus azysron, Neotropical electric fish, New Guinea rainbowfish, New World rivuline, New Zealand smelt, nibbler, Niger trigger, Nimbochromis fuscotaeniatus, Nimbochromis livingstonii, Nimbochromis polystigma, Nimbochromis venustus, noodlefish, North American darter, North American freshwater catfish, North Pacific daggertooth, northern anchovy, northern clingfish, northern lampfish, northern pearleye, northern pike, northern sea robin, northern squawfish, northern Stargazer, Northern Wobbegong Shark, Norwegian Atlantic salmon, Novaculichthys taeniourus, Nurse Shark, nurse shark, nurseryfish, oarfish, Oblique-lined Dottyback, ocean perch, ocean sunfish, oceanic flyingfish, oceanic whitetip shark, Ocellate damsel, Ochre-Striped Cardinalfish, Odonus niger, Ogilbyina novaehollandiae, oilfish, Old World knifefish, Old World rivuline, oldwife, olive flounder, Ompok bimaculatus, One Spot Blenny, One Spot Foxface, Onespot barb, opah, opaleye, Ophioblennius atlanticus, Opistognathus aurifrons, Opistognathus lonchurus, Opistognathus rosenblatti, Opistognathus whitehurstii, Opthalmotilapia ventralis, orange cheek pleco, Orange chromide, orange roughy, Orange Shoulder tang, Orange Spot Grouper, Orange Spotted goby, Orange Stripe Prawn goby, Orange-back Angelfish, Orange-Back Fairy wrasse, Orange-Striped Cardinalfish, Orangehead Anthias, Orangemarked goby, orangespine unicorn fish, Orangespotted Filefish, orangestriped triggerfish, Orangetail damsel, Orangetail Dottyback, orbicular batfish, orbicular velvetfish, Orbiculate Batfish, Orchid dottyback, Orectolobus maculatus, Orectolobus ornatus, Orectolobus wardi, Oregon chub, Oriental loach, Oriental Sweetlips, Ornate Bichir, Ornate Wobbegong Shark, Oryzias latipes, Oryzias curvinotus, Oscar, Osphronemus goramy, Ostracion cubicus, Ostracion solorensis, Otocinclus affinis, Otocinclus cocama, Owens pupfish, Oxycirrhites typus, Oxymonacanthus longirostris, Pacific albacore, Pacific argentine, Pacific cod, Pacific Double Saddle Butterflyfish, Pacific hake, Pacific herring, Pacific lamprey, Pacific Pygmy Angelfish, Pacific salmon, Pacific saury, Pacific trout, Pacific viperfish, paddlefish, Painted Comber, Painted Sweetlips, Pajama Cardinalfish, Pale usisya aulonocara, Paletail Chromis, Panaque, Panaque spp., Panda corydoras, Panda dwarf cichlid, panga, Pangasius hypophthalmus, Pangasius sanitwongsei, Pangio kuhlii, Panther Grouper, paperbone, Papuan Scorpionfish, Paracanthurus hepatus, Paracheilinus carpenteri, Paracheilinus mccoskeri, Paracheirodon axelrodi, Paracheirodon innesi, Paracheirodon simulans, Parachromis dovii, Paracirrhites arcatus, Paracirrhites forsteri, Paracirrhites hemistictus, Paracirrhites xanthus, Paradise fish, paradise fish, Paraglyphidodon oxyodon, Paraluteres prionurus, Parambassis ranga, Paramonacanthus japonicus, Parapterois heterura, Parascorpaena mossambica, parasitic catfish, Parrotfish, parrotfish, Partipentazona barb, Parupeneus barberinoides, Parupeneus barberinus, Parupeneus cyclostomus, Parupeneus multifasciatus, Passer Angelfish, Pastaza corydoras, Pavo damsel, Peacock bass, Peacock Flounder, peacock flounder, peamouth, Pearl danio, pearl danio, Pearl gourami, pearl perch, pearleye, pearlfish, pejerrey, peladillo, pelagic cod, pelican eel, pelican gulper, Pelvicachromis pulcher, pencil catfish, pencilfish, pencilsmelt, Penguin tetra, Pentazona barb, Peppered corydoras, Peppered Moray, perch, Percula Clown, Periophthalmus argentilineatus, Periophthalmus barbarus, Personifer Angelfish, Pervagor melanocephalus, Pervagor spilosoma, Peter's elephantnose fish, Petrochromis sp. Zaire Texas, Petrochromis trewavasae, Phenacogrammus interruptus, Pholidichthys leucotaenia, Pholidochromis cerasina, Phoxinus erythrogaster, Phractocephalus hemioliopterus, Picasso trigger, pickerel, pictus, Pictus catfish, pigfish, pike, pike characid, pike conger, pike eel, pikeblenny, pikehead, pikeperch, pilchard, pilot fish, Pimelodus pictus, Pimephales promelas, Pineapple pleco, pineconefish, Pink corydoras, pink salmon, Pink Skunk Anemonefish, Pink smith damsel, Pinkbar goby, Pinkface wrasse, Pinkspotted Shrimp Goby, Pinktail trigger, Pipefish, pipefish, piranha, pirarucu, pirate perch, plaice, Platax orbicularis, Platax pinnatus, Platax teira, Platies, platy, Platydoras costatus, platyfish, Platyrhinoidis triseriata, pleco, Plectorhinchus albovittatus, Plectorhinchus chaetodonoides, Plectorhinchus diagrammus, Plectorhinchus lineatus, Plectorhinchus orientalis, Plectorhinchus picus, Plectropomus laevis, plownose chimaera, plunderfish, poacher, Poecilia latipinna, Poecilia reticulata, Poecilia sphenops, Poecilia wingei, Poeciliids, Pogonoperca punctata, Polka dot syno, Polleni Grouper, pollock, pollyfish, Polypterus senegalus, Pomacanthus annularis, Pomacanthus arcuatus, Pomacanthus asfur, Pomacanthus imperator, Pomacanthus maculosus, Pomacanthus navarchus, Pomacanthus paru, Pomacanthus semicirculatus, Pomacanthus zonipectus, Pomacentrus alleni, Pomacentrus amboinensis, Pomacentrus auriventris, Pomacentrus bankanensis, Pomacentrus caeruleus, Pomacentrus coelestis, Pomacentrus moluccensis, Pomacentrus nigromarginatus, Pomacentrus pavo, Pomacentrus simsiang, Pomacentrus smithi, Pomacentrus vaiuli, pomfret, pompano, pompano dolphinfish, ponyfish, Pool barb, poolfish, poor man's tropheus, popeye catafula, Popeye Catalufa Soldierfish, porbeagle shark, Porcupine Pufferfish, porcupinefish, porgy, Porkfish, Port Jackson Shark, Port Jackson shark, Poss's Scorpionfish, Pot-Bellied Seahorse, Potter's Angelfish, Potter's wrasse, Powder blue tang, Powder Brown Tang, powen, Premnas biaculeatus, pretty tetra, priapumfish, prickleback, pricklefish, prickly shark, Princess Parrotfish, Priolepis aureoviridis, Priolepis nocturnal Pristigenys serrula, Protomelas taeniolatus, prowfish, Pseudanthias bartlettorum, Pseudanthias bicolor, Pseudanthias cooperi, Pseudanthias heemstrai, Pseudanthias huchtii, Pseudanthias hypselosoma, Pseudanthias parvirostris, Pseudanthias pleurotaenia, Pseudanthias rubrizonatus, Pseudanthias squamipinnis, Pseudobalistes fuscus, Pseudocheilinus evanidus, Pseudocheilinus hexataenia, Pseudocheilinus ocellatus, Pseudocheilinus octotaenia, Pseudocheilinus tetrataenia, Pseudochromis, Pseudochromis aldabraensis, Pseudochromis aureus, Pseudochromis bitaeniatus, Pseudochromis coccinicauda, Pseudochromis cyanotaenia, Pseudochromis diadema, Pseudochromis dilectus, Pseudochromis elongatus, Pseudochromis flammicauda, Pseudochromis flavivertex, Pseudochromis fridmani, Pseudochromis fuscus, Pseudochromis paccagnellae, Pseudochromis porphyreus, Pseudochromis sankeyi, Pseudochromis splendens, Pseudochromis springeri, Pseudochromis steenei, Pseudochromis veliferus, Pseudoplatystoma fasciatum, Pseudoplatystoma tigrinum, Pseudorinelepis spp., Pseudotropheus acei, Pseudotropheus crabro, Pseudotropheus saulosi, Pterapogon kauderni, Ptereleotris evides, Ptereleotris hanae, Ptereleotris zebra, Pteroidichthys amboinensis, Pterois antennata, Pterois mombasae, Pterois radiata, Pterois russelli, Pterois volitans, Pterophyllum altum, Pterophyllum leopoldi, Pterophyllum scalare, Pterygoplichthys gibbiceps, Pterygoplichthys multiradiatus, Pterygoplichthys pardalis, Ptyochromis sp. Hippo Point Salmon, Pufferfish, pufferfish, pumpkinseed, Pundamilia nyererei, Puntius arulius, Puntius binotatus, Puntius chola, Puntius conchonius, Puntius denisonii, Puntius dunckeri, Puntius everetti, Puntius fasciatus, Puntius gelius, Puntius lateristriga, Puntius nigrofasciatus, Puntius oligolepis, Puntius partipentazona, Puntius pentazona, Puntius phutunio, Puntius semifasciolatus, Puntius sophore, Puntius stoliczkanus, Puntius terio, Puntius tetrazona, Puntius titteya, Puntius vittatus, pupfish, Purple Chromis, Purple Fire Fish, purple psuedochromis, Purple stripe pseudochromis, Purple tang, Purple tilefish, Pygmy Angelfish, Pygmy corydoras, Pygmy gourami, Pygmy Seahorse, pygmy sunfish, Pygoplites diacanthus, pintano, Queen Angelfish, Queen danio, queen danio, queen parrotfish, Queen trigger, queen triggerfish, quillback, quillfish, Rabbitfish, rabbitfish, raccoon butterfly fish, Raccoon Butterflyfish, Radiant Wrasse, Radiata lionfish, ragfish, rainbow trout, Rainbowfish, rainbowfish, rams, rasbora, Rasbora borapetensis, Rasbora pauciperforata, Rasbora vulcanus, Rasboras, ratfish, rattail, ray, Rays, razorback sucker, razorfish, Rectangular trigger, Red bellied pacu, Red Coris Wrasse, Red devil cichlid, red devil cichlid, Red Diana Hogfish, red discus, Red Dottyback, Red empress cichlid, Red Flag Grouper, Red Head goby, Red line torpedo barb, Red Lip Blenny, Red Scooter Blenny, Red Sea Mimic Blenny, red snapper, Red Striped goby, Red Velvet Wrasse, red velvetfish, red whalefish, Red zebra cichlid, Red-finned shark, Red-head fairy wrasse, Red-striped rasbora, Red-tailed black shark, red-tailed rasbora, Red-top kimpumpa, Redback Butterflyfish, Redbar Anthias, redfin perch, redfish, redhorse sucker, Redlined tilefish, redlip blenny, redmouth whalefish, redside, Redspotted hawkfish, Redtail catfish, redtooth triggerfish, Reedfish, reedfish, reef triggerfish, regal whiptail catfish, remora, requiem shark, reticulated corydoras, Rhampsochromis cf macrophthalmus, Rhinecanthus aculeatus, Rhinecanthus assasi, Rhinecanthus rectangulus, Rhinecanthus verrucosus, Rhinomuraena quaesita, Rhinopias aphanes, Rhinopias eschmeyeri, Rhinopias frondosa, Rhomboid Fairy Wrasse, ribbon eel, ribbon sawtail fish, ribbonbearer, ribbonfish, rice eel, ricefish, ridgehead, riffle dace, righteye flounder, Rineloricaria spp., Ringtailed Cardinalfish, Rio Grande cichlid, Rio Grande perch, river loach, river shark, river stingray, rivuline, roach, roanoke bass, rock bass, rock beauty, rock cod, rocket danio, rockfish, rockling, rockweed gunnel, Rogue Scorpion, rohu, Rolland's demoiselle, ronquil, roosterfish, ropefish, Rosy barb, Rosy red minnow, rough pomfret, rough scad, rough sculpin, roughy, round herring, Round stingray, round stingray, round whitefish, roundhead, Royal Angelfish, Royal Gramma, Ruby Scat, rudd, rudderfish, ruffe, Rummy-nose tetra, Russell's lionfish, Russian sturgeon, Rusty Angelfish, Rusty cichlid, sabalo, saber-toothed blenny, sabertooth, sabertooth fish, sablefish, Sacramento blackfish, Sacramento splittail, Saddle Grouper, Saddleback Anemonefish, sailback scorpionfish, sailbearer, Sailfin Blenny, Sailfin catfish, Sailfin corydoras, Sailfin molly, Sailfin pseudochromis, sailfin silverside, Sailfin tang, sailfish, salamanderfish, Salarias fasciatus, Salarias ramosus, Salarias segmentatus, salmon, salmon shark, Salmon-red rainbowfish, Salt and pepper catfish, sand dab, sand eel, sand goby, sand knifefish, sand lance, sand stargazer, sand tiger, sand tilefish, sandbar shark, sandburrower, sanddiver, sandfish, sandperch, sandroller, sarcastic fringehead, sardine, sargassum fish, Sargassum Frogfish, Sargassum trigger, Sargocentron tiere, Sargocentron xantherythrum, Sarotherodon melanotheron melanotheron, sauger, Saulosi, saury, saw shark, sawfish, sawtooth eel, scabbard fish, scaleless black dragonfish, scaly dragonfish, Scarlet Pin Stripe Wrasse, Scarlet Squirrelfish, Scarus taeniopterus, scat, Scatophagus argus, Schwartz's catfish, scissor-tail rasbora, Scissortail Dartfish, Scleromystax barbatus, Scooter Blenny, Scopas tang, Scorpaenopsis macrochir, Scorpaenopsis papuensis, Scorpaenopsis possi, Scorpionfish, scorpionfish, Scribbled Angelfish, Scribbled Boxfish, sculpin, scup, scythe butterfish, sea bass, Sea catfish, sea catfish, sea chub, Sea Goblin, sea lamprey, sea raven, sea snail, sea toad, seadevil, seadragon, Seahorse, seahorse, seamoth, searobin, seatrout, Sebae Anemonefish, Sebastapistes cyanostigma, Segmented Sailfin Blenny, Selene vomer, sergeant major, Sergeant major damsel, Serpae tetra, Serranus scriba, Serranus tigrinus, Serranus tortugarum, Sevan trout, Severum, shad, shark, Sharks, sharksucker, Sharpnose Pufferfish, sharpnose pufferfish, sheatfish, sheepshead, sheepshead minnow, shell-ear, shiner, Short-Snouted Seahorse, Shortfin barb, shortnose chimaera, shortnose greeneye, shortnose sucker, shovelnose sturgeon, shrimpfish, Shy Hamlet, Siamese algae eater, Siamese fighting fish, Siamese tigerfish, Sickle Butterflyfish, Siganus doliatus, Siganus guttatus, Siganus magnificus, Siganus spp., Siganus unimaculatus, Siganus uspi, Siganus vulpinus, Signigobius biocellatus, sillago, silver carp, Silver dollar, silver dollar, silver driftfish, silver hake, Silver hatchetfish, Silver moony, silverside, Simochromis pleurospilus, Sind danio, sind danio, Six line wrasse, Sixband barb, sixgill ray, sixgill shark, Sixray corydoras, skate, skilfish, skipjack tuna, skipping goby, Skunk loach, sleeper, Sleeper Banded goby, Sleeper Blue Dot goby, Sleeper Gold Head goby, Sleeper Railway Glider goby, sleeper shark, Sleeper Striped goby, slender barracudina, slender mola, slender snipe eel, slickhead, slimehead, slimy mackerel, slimy sculpin, slipmouth, smalleye squaretail, smalltooth sawfish, smelt, smelt-whiting, smooth dogfish, smoothtongue, snailfish, snake eel, snake mackerel, snake mudhead, snakehead, Snakeskin gourami, snapper, Snappers, snipe eel, snipefish, snoek, snook, Snowflake eel, snubnose eel, snubnose parasitic eel, soapfish, sockeye salmon, Sohal tang, soldierfish, sole, Soleichthys heterorhinos, sora goldfish, South American darter, South American Lungfish, southern flounder, southern grayling, southern hake, Southern platy, Southern redbelly dace, southern sandfish, southern smelt, spadefish, spaghetti eel, Spanish Hogfish, Spanish mackerel, Spanner barb, Sparkling gourami, spearfish, Speckled damsel, speckled trout, Sphaeramia nematoptera, Sphaerichthys osphromenoides, spiderfish, spikefish, Spilo, spinefoot, spiny basslet, Spiny Chromis, spiny dogfish, spiny dwarf catfish, spiny eel, Spiny eels, spiny-back, spinyfin, Spinyhead Blenny, Splendid pseudochromis, splitfin, spookfish, Spotfin Porcupinefish, Spotfinned spiny eel, Spotted angelfish, Spotted barb, Spotted Caribbean stingray, Spotted corydoras, Spotted danio, spotted danio, spotted dogfish, Spotted Gar, Spotted Garden-Eel, Spotted Grouper, Spotted hawkfish, Spotted Mandarin, Spotted pimelodus, Spotted Raphael, Spotted Seahorse, Spotted Snake Eel, Spotted Sweetlips, Spotted Wobbegong Shark, Spottedsail barb, sprat, Springer's damsel, Springeri pseudochromis, springfish, Squareback anthias, squarehead catfish, squaretail, squawfish, squeaker, Squirrelfish, squirrelfish, staghorn sculpin, Star Puffer, stargazer, Stark's damsel, Starry Blenny, starry flounder, Starry Trigger, Stars and Stripes Puffer, Steatocranus, steelhead, Stegastes diencaeus, Stegastes planifrons, Steinitz goby, Sterba's corydoras, Steve fish, stickleback, Stigmatogobius sadanundio, stingfish, stingray, Stocky Anthias, Stone Fish, stonecat, stonefish, stoneroller minnow, Stonogobiops dracula, Stonogobiops nematodes, Stonogobiops yasha, straptail, Strawberry Grouper, Strawberry pseudochromis, stream catfish, streamer fish, Striated frogfish, striped bass, Striped Blenny, striped burrfish, Striped Dogface Puffer, Striped dottyback, Striped Raphael, Striped Squirrelfish, Striped Sweetlips, sturgeon, sucker, suckermouth armored catfish, suckermouth catfish, Sufflamen albicaudatum, Sufflamen bursa, Sufflamen chrysopterum, sumatra barb, Sumireyakko venustus, summer flounder, Sundaland noodlefish, sunfish, Sunshine Chromis, Sunshine peacock cichlid, surf sardine, surfperch, surgeonfish, swallower, Swamp barb, swamp-eel, swampfish, sweeper, swordfish, swordtail, swordtails, Sychr's catfish, Symphorichthys spilurus, Symphysodon aequifasciatus, Symphysodon discus, synanceja verrucosa, Synchiropus ocellatus, Synchiropus picturatus, Synchiropus splendidus, Synchiropus stellatus, Synodontis angelicus, Synodontis decorus, Synodontis eupterus, Synodontis multipunctatus, Synodontis nigrita, Synodontis nigriventris, Synodontis njassae, Synodontis petricola, T-bar cichlid, t-barb, tadpole cod, tadpole fish, Taenianotus triacanthus, Taenioconger hassi, Tail Spot Blenny, tailor, Tailspot corydoras, taimen, Talbots damsel, Tami River rainbowfish, tang, Tangaroa goby, Tangs, Tanichthys albonubes, tapetail, Targetfish, tarpon, tarwhine, Tassle filefish, Teardrop Butterflyfish, Teira Batfish, telescopefish, temperate bass, temperate ocean-bass, temperate perch, tench, Tennent tang, tenpounder, tenuis, Terapon jarbua, Tessalata Eel, tetra, Tetraodon biocellatus, Tetraodon mbu, Tetraodon miurus, Tetraodon nigroviridis, Tetrasomus gibbosus, Texas cichlid, Thalassoma bifasciatum, Thalassoma hebraicum, Thalassoma jansenii, Thalassoma lucasanum, Thalassoma lunare, Thalassoma lutescens, Thalassoma quinquevittatum, Thalassoma trilobatum, Thayeria boehlkei, Thornback stingray, thornfish, thorny catfish, Thorny Seahorse, thornyhead, Thread-finned acara, threadfin, Threadfin Anthias, threadfin bream, Threadfin Butterflyfish, Threadfin Lookdown, Threadfin rainbowfish, Threadfin Snapper, threadsail, threadtail, Three Spot damsel, Three spot gourami, three spot gourami, Three stripe damsel, Three-lined pencilfish, Three-Stripe Dwarf Cichlid, three-toothed puffer, threespine stickleback, Threestripe corydoras, thresher shark, Tic-tac-toe barb, Ticto barb, tidewater goby, Tiger barb, tiger barb, tiger shark, Tiger shovelnose catfish, tiger shovelnose catfish, Tiger Tail Seahorse, Tiger Watchman goby, tigerperch, tilapia, Tilapia buttikoferi, Tilefish, tilefish, Tinfoil barb, Tinker's Butterflyfish, Tire track eel, Titan trigger, titan triggerfish, toadfish, Tomato Clownfish, Tomini tang, tommy ruff, tompot blenny, tonguefish, tope, topminnow, torpedo, torrent catfish, torrent fish, Toxotes jaculatrix, trahira, treefish, trevally, Trichogaster leerii, Trichogaster microlepis, Trichogaster pectoralis, Trichogaster trichopterus, Trichopsis pumila, Trichopsis vittata, Triggerfish, triggerfish, Trigonostigma heteromorpha, Trimma cana, triplefin blenny, triplespine, tripletail, tripod fish, Tropheus duboisi, Tropheus moorii, Tropheus polli, trout, trout cod, trout-perch, trumpeter, trumpetfish, trunkfish, Trygonorhina fasciata, tube-eye, tube-snout, tubeblenny, tubeshoulder, tui chub, tuna, turbot, turkeyfish, Turquoise danio, Tuxedo damsel, Twig catfish, Twin Spot Hogfish, Two spot glass catfish, Two Spot goby, Two Stripe Damsel, Two-Spot Blenny, two-spotted jewel cichlid, Two-Stripe Dwarf Cichlid, Twolined Dottyback, Twostriped Sweetlips, Tyrannochromis macrostoma, Uaru, Uaru amphiacanthoides, Undulated trigger, Unicorn tang, unicornfish, Upside-down catfish, upside-down catfish, Urobatis halleri, Urolophus spp., V Tail Grouper, Valenciennea helsdingeni, Valenciennea longipinnis, Valenciennea puellaris, Valenciennea sexguttata, Valenciennea strigata, Valenciennea wardii, Valentini Pufferfish, Variabilichromis, Variable platy, Variola louti, velvet catfish, velvet-belly shark, velvetfish, vendace, Venustus Angelfish, Venustus cichlid, vimba, Violet goby, viperfish, Vlamingi tang, Volcano rasbora, Volitan lionfish, wahoo, Walking catfish, walking catfish, wallago, walleye, walleye pollock, walu, warbonnet, warmouth, waroo, Wartskin Angler, warty angler, waryfish, wasp fish, Water Cow, weasel shark, weather loach, weatherfish, Weedy Scorpionfish, weever, weeverfish, wels catfish, whale catfish, whale shark, whalefish, Wheeler's Watchman goby, whiff, Whip Fin Fairy Wrasse, Whiptail catfish, whiptail gulper, White Cloud Mountain minnow, white croaker, White Freckled surgeon, white marlin, white shark, White's Seahorse, whitebait, Whitebelly Puffer, whitefish, Whitemouth Moray Eel, Whitespot hawkfish, Whitespotted Bambooshark, Whitetail Trigger, whitetip reef shark, whiting, wobbegong, Wolf Eel, wolf-eel, wolf-herring, wolffish, woody sculpin, worm eel, wormfish, Wrasse, wrasse, wrymouth, x-ray tetra, Xanthichthys auromarginatus, Xanthichthys caeruleolineatus, Xanthichthys mento, Xanthiethys ringens, Xanthurus Cream Angelfish, Xingu corydoras, Xiphosphorus hellerii, Xiphosphorus maculatus, Xiphosphorus variatus, Xystichromis phytophagus, Yashia goby, Yasuhikotakia modesta, Yasuhikotakia morleti, Yasuhikotakia sidthimunki, Yellow & Purple Wrasse, Yellow Assessor, Yellow Back Goatfish, yellow bass, Yellow Blotch Rabbitfish, Yellow Boxfish, Yellow clown goby, Yellow damsel, Yellow dwarf cichlid, Yellow Fin Fairy Wrasse, Yellow hawkfish, yellow jack, yellow moray, Yellow Mouth Moray Eel, Yellow Multibanded Pipefish, yellow perch, Yellow Priolepis goby, Yellow Pseudochromis, Yellow Stripe Clingfish, Yellow tang, yellow tang, Yellow Threespot dascyllus, Yellow Watchman goby, yellow weaver, Yellow Wrasse, yellow-and-black triplefin, yellow-edged moray, yellow-eye mullet, Yellow-Flanked Fairy Wrasse, Yellowback Fusilier, Yellowband Wrasse, yellowbanded perch, Yellowbelly damsel, yellowfin croaker, yellowfin cutthroat trout, Yellowfin demoiselle, yellowfin grouper, yellowfin pike, Yellowfin surgeon, yellowfin surgeonfish, yellowfin tuna, Yellowhead jawfish, yellowhead jawfish, Yellowhead Moray Eel, yellowmargin triggerfish, Yellowspotted Chromis, Yellowspotted Scorpionfish, yellowtail, yellowtail amberjack, yellowtail barracuda, yellowtail clownfish, Yellowtail damsel, Yellowtail dascyllus, Yellowtail demoiselle, yellowtail horse mackerel, yellowtail kingfish, yellowtail snapper, Yoyo loach, zander, Zebra Barred Dartfish, zebra bullhead shark, Zebra danio, zebra danio, Zebra dwarf sucker, Zebra lionfish, zebra lionfish, Zebra loach, zebra loach, Zebra moray, Zebra obliquidens, zebra oto, Zebra pleco, zebra pleco, zebra shark, Zebra tilapia, zebra tilapia, zebrafish, Zebrasoma desjardinii, Zebrasoma flavescens, Zebrasoma scopas, Zebrasoma veliferum, Zebrasoma xanthurus, ziege, and zingel.

The invention will now be described in more detail with respect to the following, specific, non-limiting examples.

### EXAMPLES

### Example 1

### Cloning of Golden and GFP-Expressing Zebrafish

Animals of homozygous golden zebrafish strain (slc24a5^{b1/b1}) (11) with AB background display golden phenotypes while heterozygous animals appear as wild-type, facilitating phenotypic screening of clones. In addition, to demonstrate the broad applicability of this technique to other strains, we cloned transgenic fish expressing green fluorescence protein (HGn62A, HGn28A and HGn8E) (12) with Tuebingen—long fin background (kindly donated by Dr. Kawakami). We tested two primary sources of donor cells which were either freshly isolated cells from the tail-bud of an embryo at 15-20 somite-stage (ET) or cultured fibroblasts from adult caudal fin (AF). Recipient eggs were obtained from wild-type, transgenic homozygous histone H2A-tagged green fluorescent protein (H2AzGFP) with AB background fish (13), or outcrossed of Tuebingen and AB line (TAB). The use of golden donor cells in combination with wild-type pigmented pattern of recipient eggs simplified the initial verification of cloned fish produced, that is, a pigmented pattern in cloned fish can be monitored via a stereoscope. The cloned fish of transgenic Tuebingen—long fin donor cells can easily be verified by expression of GFP in cloned embryos as well as a long fin phenotype of adult fish. In addition, the use of transgenic H2AGFP^{+/+} as well as SNP analysis provided supporting evidence of complete inactivation of the egg genome by the loss of nuclear localized GFP in cloned fish and the complete matched DNA fingerprinting of cloned fish to one of the donor cells, respectively.

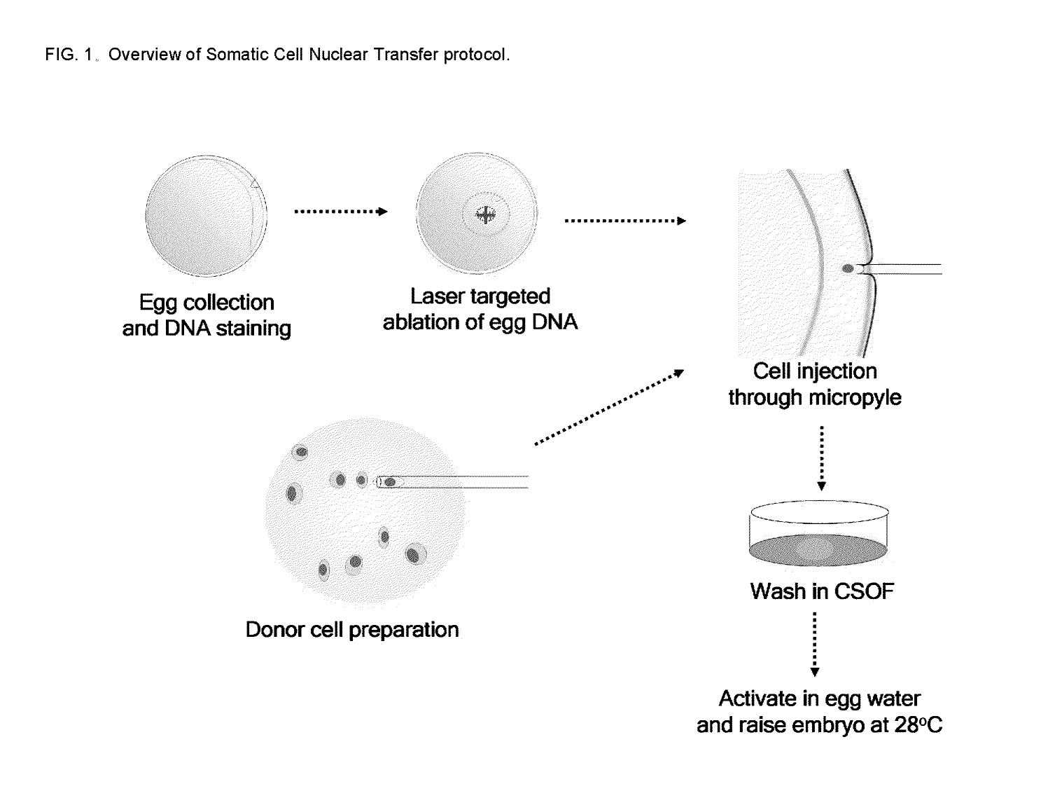

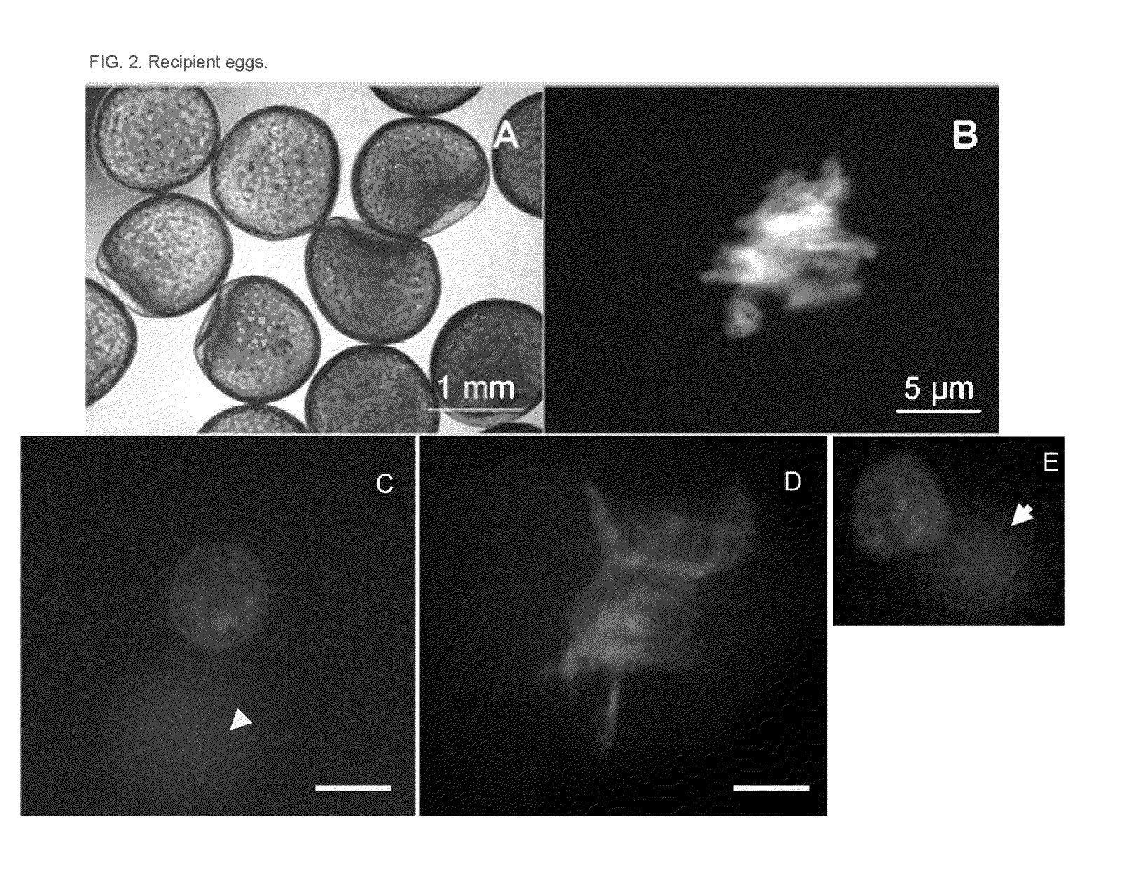

Nuclear transfer (NT) was performed using the general procedures outlined in FIG. 1, using one pipette for holding a recipient egg and double injection needles for support and nuclear transfer. Nuclear transfer was performed using one pipette for holding the recipient egg and double injection needles for supporting and nuclear transfer. The egg holding pipette was cut straight and fire-polished to 200-300 μm inner diameter. A Human ICSI needle (Humangen, Charlottesville, Va.) was used for nuclear transfer. For embryonic cells, needles with inner diameter of 5-6 μm were used. For cultured adult cells, needles with inner diameters of 8-9 μm were used. The supporting needle (inner diameter 20 μm) was set up in parallel with the injection needle to help rotate the egg.

Eggs were collected in Chinook salmon ovarian fluid (CSOF) and stained with Hoechst 33342 (FIGS. 2A-2B). Drops of 5% PVP in CSOF were used as a manipulation medium. Approximately five eggs were manipulated at a time. Manipulation time preferably does not exceed 4 hours post egg collection, however, conditions (such as temperature, manipulation medium, strain or species used, etc.) may be optimized to permit longer manipulation if desired. Cells were treated with HOECHST to stain the DNA and allow its visualization; depending on the dye and irradiation source used, a stain may also increase sensitivity of the nucleus to laser ablation. As noted above, other techniques may be employed to visualize the nucleus, rendering DNA staining potentially optional. A laser assisted XYClone module (Hamilton Thorne Biosciences, Inc.) was used for targeted ablation of metaphase plate of the recipient egg. Laser exposure was performed using a 40× laser objective lens and controller. The egg was positioned with the micropyle facing the bottom of a manipulation dish, allowing the metaphase plate to be visualized best under UV light. The metaphase plate was moved to a laser target and exposed/irradiated for 500 μS at 100% power approximately 2-4 times. Complete ablation is confirmed by nuclear staining of parthenogenetically activated egg at 20 min after laser-ablation, which shows complete inactivation of egg DNA and no extrusion of a second polar body (FIG. 2D; scale bar is 10 μm). In contrast, Nuclear staining of parthenogenetically activated control eggs at 20 min after egg-activation indicates female pronuclear formation and complete extrusion of second polar body (FIGS. 2C and 2E, arrow; scale bar is 10 μm).

Donor cells, placed in 2% PVP in serum-depleted D-NACs, were loaded into the ICSI needle. Since the internal diameter of the needle used was slightly smaller than the cell, it was used to break the cell membrane while leaving the nucleus intact. The recipient egg was repositioned with its micropyle now facing the injection needle, so that the donor nucleus and its remaining cytosol could then be transferred to the animal pole of the egg via the micropyle. The ICSI needle was small enough to allowed transfer of the donor nucleus directly into an animal pole of the egg through the micropyle. Anatomically, the micropyle is located at the animal pole of the egg, providing a good landmark for transferring the donor nuclei while circumventing the need for removing the chorion, a step that required the use of pronase and leads to premature activation of the egg (15).

The reconstructed embryos were washed in CSOF for 15 minutes, and subsequently activated in egg water (60 μg/ml sea salt) and incubated at 28° C. The development of cloned embryos was monitored and recorded at blastula (3 hr), germ ring (6 hr), 90% epiboly (9-10 hr), and daily thereafter until reaching adulthood.

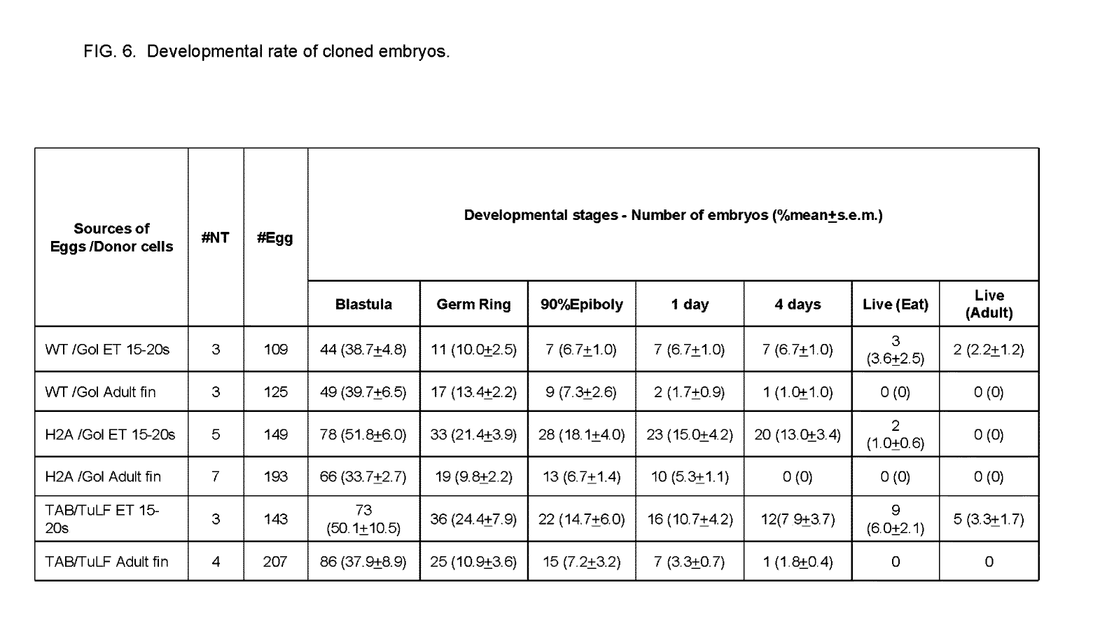

SCNT operations were performed using wild-type eggs (WT), transgenic H2Az-GFP eggs (H2A), or Tuebigen-AB outcrossed eggs (TAB). Donor cells were isolated from either freshly dissociated tail-buds of embryos at 15-20 somites (ET) or cultured of adult fin fibroblasts (AF) of homozygous golden strain (Gol), or Tuebingen-long fin (TuLF). Number of total NT operations (#NT). Number of total eggs in each operation (#Egg). Donor cells were derived from either cultured adult fin (“Adult fin”) fibroblasts or freshly isolated cells from tail-buds of embryos at 15-20 somites (“15-20s”). Referring now to FIG. 6, numbers indicate total eggs or cloned embryos (% mean±standard error) in each experimental group. Approximately 1% to 13% of cloned hatch fry were obtained from donor cells derived from cultured adult fibroblasts and embryonic cells, respectively.

To verify our manipulation technique, we produced zebrafish ICSI embryos by injecting sperm nuclei into ‘off-target’ laser-treated eggs (ablated location adjacent to the metaphase plate sparing the egg's DNA). We obtained approximately 5% adult fish per total eggs manipulated using the ICSI technique, suggesting that it should also work for SCNT.

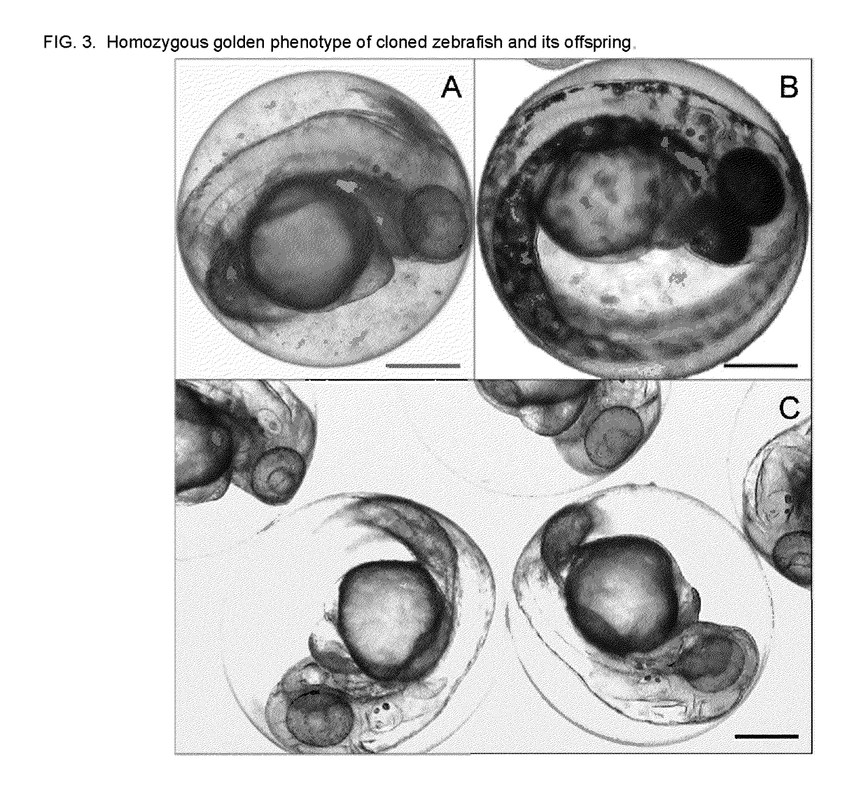

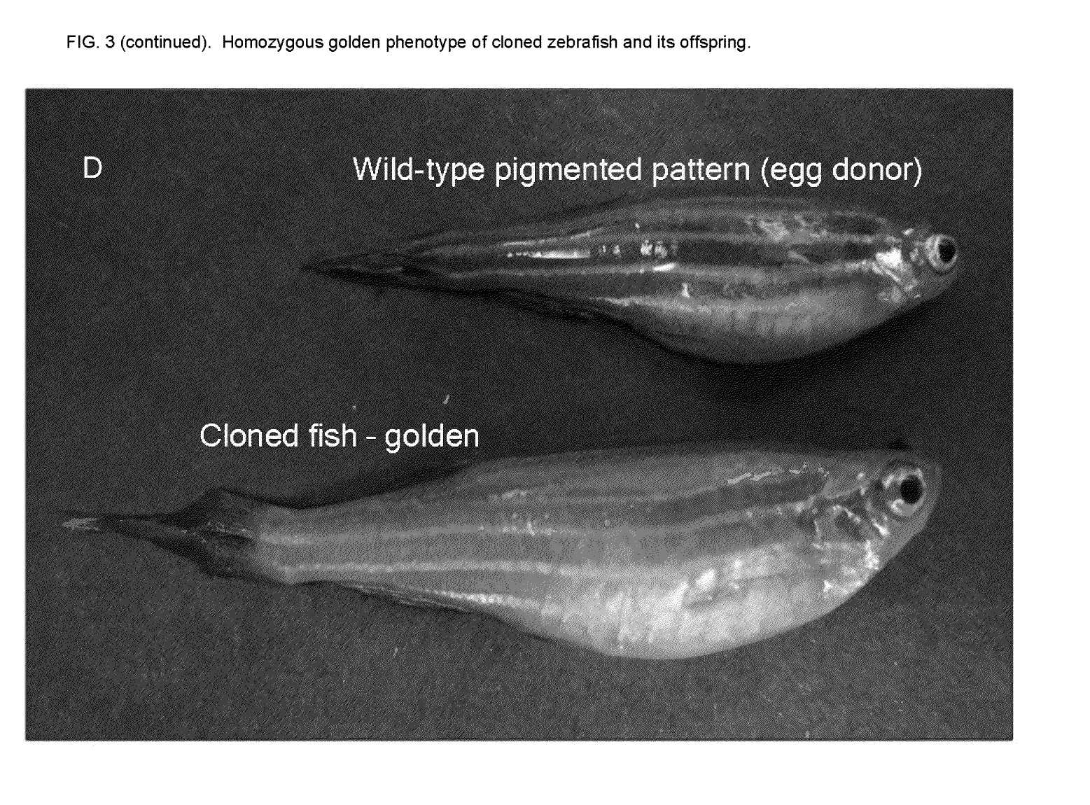

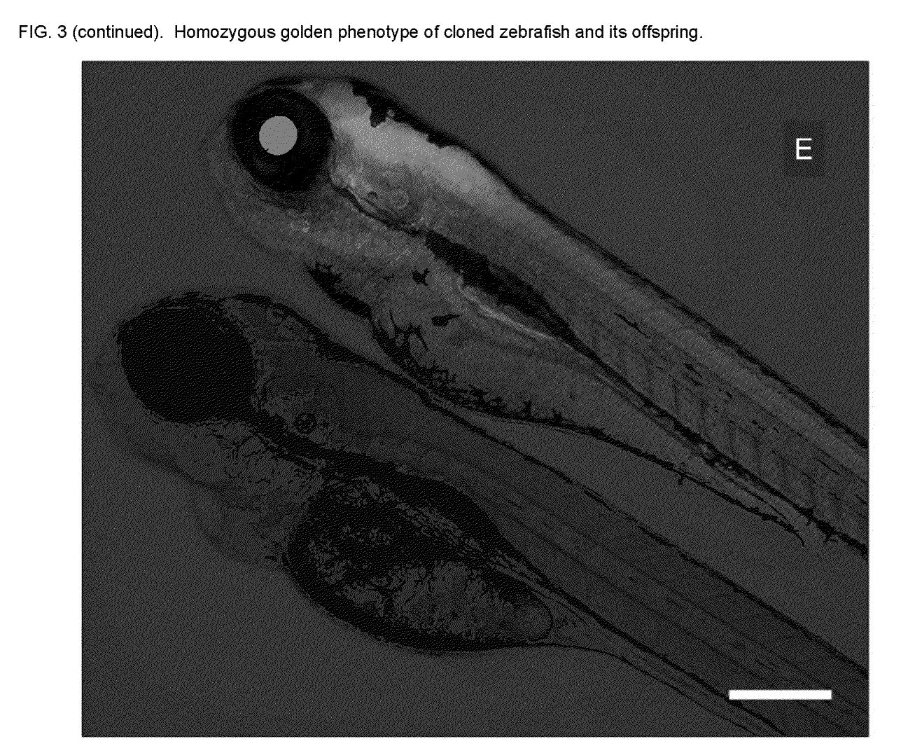

Using donor cells of a golden strain, approximately 2-15% of reconstructed embryos developed to 1 day-old fry, depending on the source of donor nuclei used (FIG. 6). All cloned embryos possessed golden phenotypes. Up to 2.2% of cloned embryos from total eggs manipulated, using ET donor cells and wild-type recipient eggs, grew to fertile adult fish (FIG. 6). Clones from golden fish at reproductive maturity, when crossbred with a golden counterpart, produced 100% golden offspring (FIG. 3D). Offspring (F1) of both golden clones were fertile and produced golden F2 generation. At the time of this writing, one of adult golden-cloned fish was 20 month old and healthy (FIG. 3B). The other adult golden-cloned fish died at 16 month of age.

For the donor cells of transgenic Tuebingen strains, approximately 3.3-10.7% of reconstructed embryos developed to 1 day-old fry (FIG. 6), depending on the source of donor nuclei used. When using ET donor cells, as many as 6% developed to eating fry and five clones survived to adulthood. All of the cloned embryos expressed green fluorescence protein with the same pattern as their donor cells. At the time of this writing, 3 cloned fish were at 3 month of age, healthy and produced offspring that carries their genetic traits.

We observed that approximately 40% of reconstructed embryos completed blastula stage (FIG. 6). Of these, more than half paused between high (3 hr) and sphere (4 hr) stage, and later on failed to enter gastrulation. Cloned embryos that failed to complete gastrulation (90% epiboly), usually completed germ ring stage but failed to form embryonic shield, an involution of hypoblast. Most of cloned embryos from ET donors that completed 90% epiboly developed to 1 day (completed segmentation). For AF donors, less than half of embryos that completed gastrulation also completed segmentation

All of the cloned fish derived from homozygous golden donor nuclei were golden phenotype. Cloned fish that reached their reproductive maturity were fertile and produced golden offspring. For example, cloned embryos at 2 days of age (FIG. 3A) possessed golden pigmented pattern which is different from wild-type fertilized embryo of same age (FIG. 3B). Offspring of adult female cloned zebrafish and male homozygous golden counterpart were also golden (FIG. 3C-3D), with adult golden cloned fish showing lack of heavy pigments that can be distinguished from the wild-type pigmented pattern of female egg-donor. As expected, a cloned golden embryo at 3 days of age shows no expression of green fluorescence protein, unlike an in vitro fertilized embryo of the same female H2AzGFP egg-donor (FIG. 3E). Scale bars are 0.5 mm.

This method allows for longer manipulations sessions, with a throughput of approximately 50 eggs per person per day. Manipulating eggs with intact chorion makes them more tolerant to micromanipulation and injection.

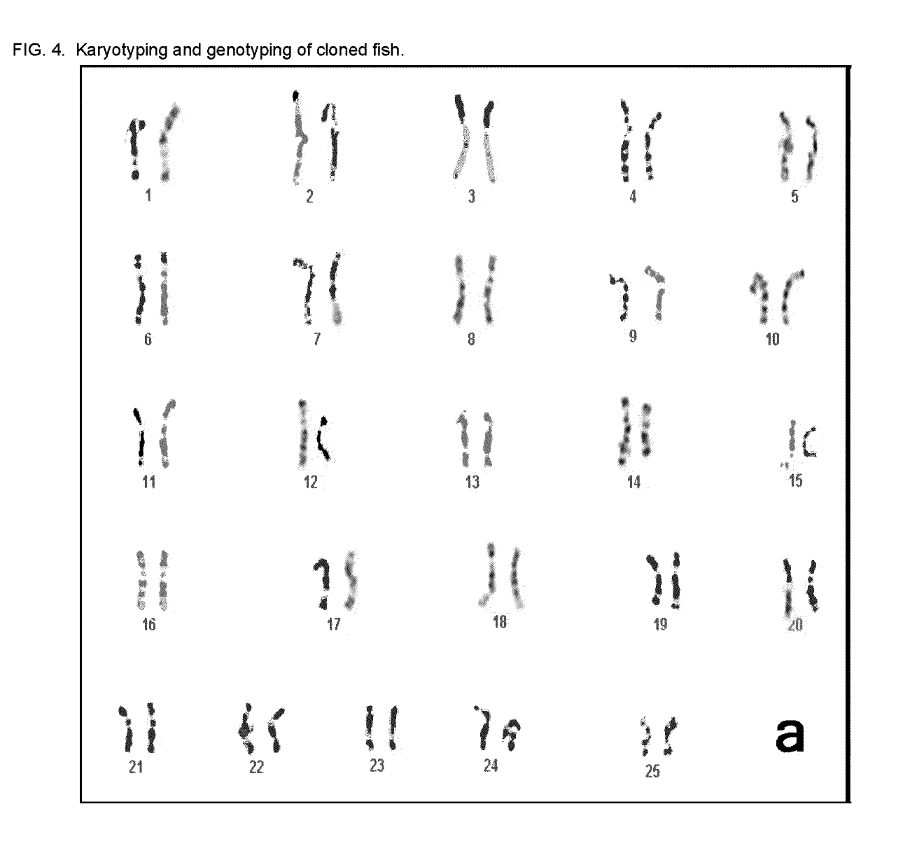

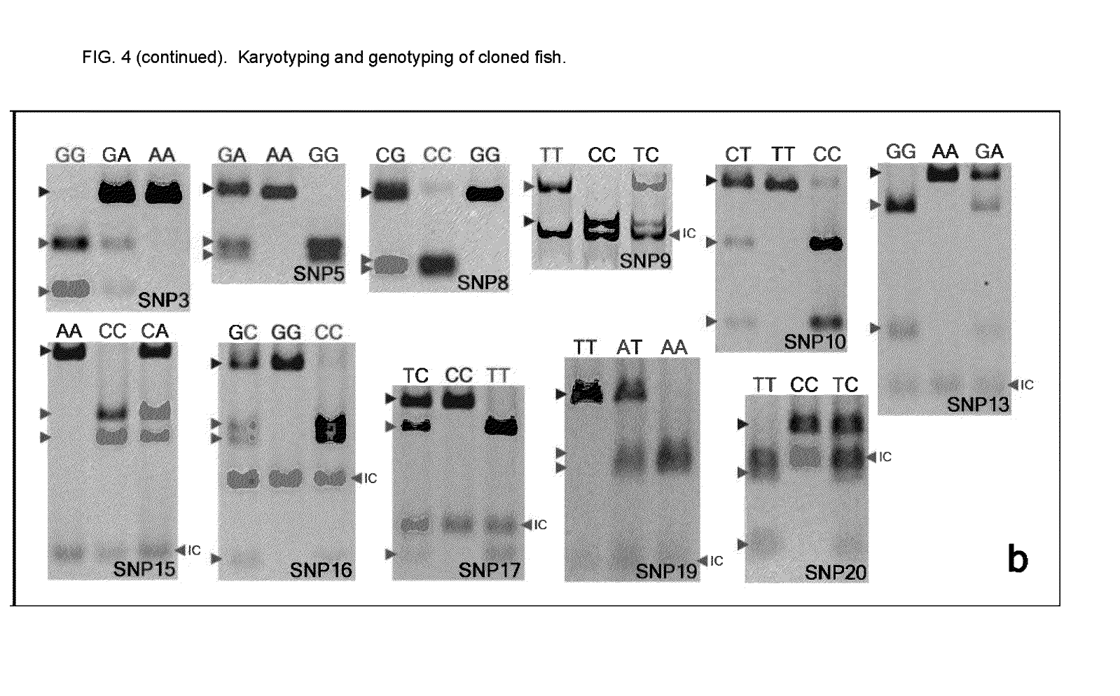

Twenty-four hours post nuclear transfer, we observed various degrees of abnormalities in reconstructed embryos (FIG. 4). Some had subtle phenotypic changes, while others had impaired development of the swim bladder, and still others had severe defects in development which manifested as a lack of development of the body. Most abnormal clones showed more posterior rather than anterior developmental defects, that is, all abnormal embryos showed primitive development of the head and eyes but defective tail formation. Abnormalities in hematopoiesis and enlarged pericardial cavity were also found (FIG. 4C). Abnormal clones derived from AF donors showed more progressive abnormalities than ones derived from ET donors. Interestingly, we did not observe a ‘no head’ phenotype as reported by Lee, et al. (3).

Most of the cloned embryos that did not develop to 4 day-old displayed severe abnormalities at one day of age such as growth retardation, bend tail, small head, and short trunk. The cloned embryos that developed to 4 day-old but failed to eat, showed minimal abnormalities including no swim bladder formation, enlarge pericardium; some of them had undetectable abnormal phenotype but died after 7-10 days. Some cloned embryos, even though they were capable of eating by themselves, died at the age of 12-20 days lacking any obvious abnormalities. Referring now to FIG. 4, illustrated are abnormal cloned embryos derived from embryonic cells at 1 day (FIGS. 4A-4B) and 3 days (FIG. 4C) of age, and cultured adult fibroblasts at 1 day (FIGS. 4D-4F). Scale bars are 0.5 mm. Most of the abnormal cloned embryos showed severe abnormalities of posterior development, and mild to moderate changes of anterior parts.

### Example 2

### Karyotyping and Genotyping of Cloned Fish

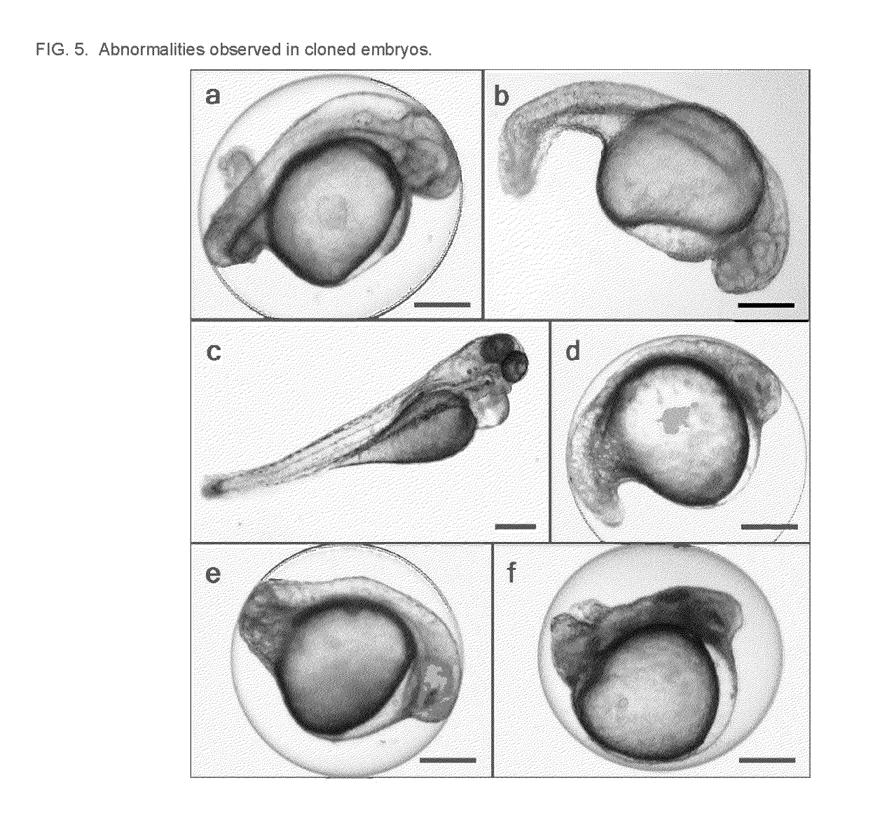

Cultured cells derived from caudal fin of cloned fish were expanded and prepared for karyotyping by replication(R) banding. R-banding was chosen because it provides substantial resolution to identify different chromosomes of zebrafish (10). All cloned fish examined had a normal karyotype. An exemplary R-banding result, shown in FIG. 5A, demonstrates the normal diploid karyotype of cloned zebrafish (2n=50).

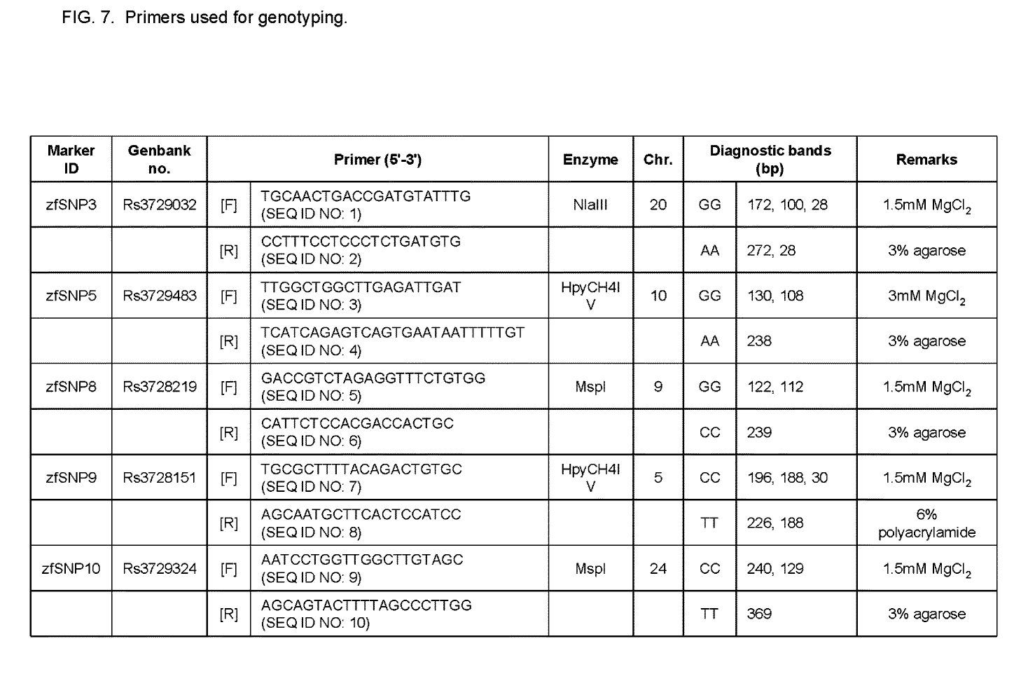

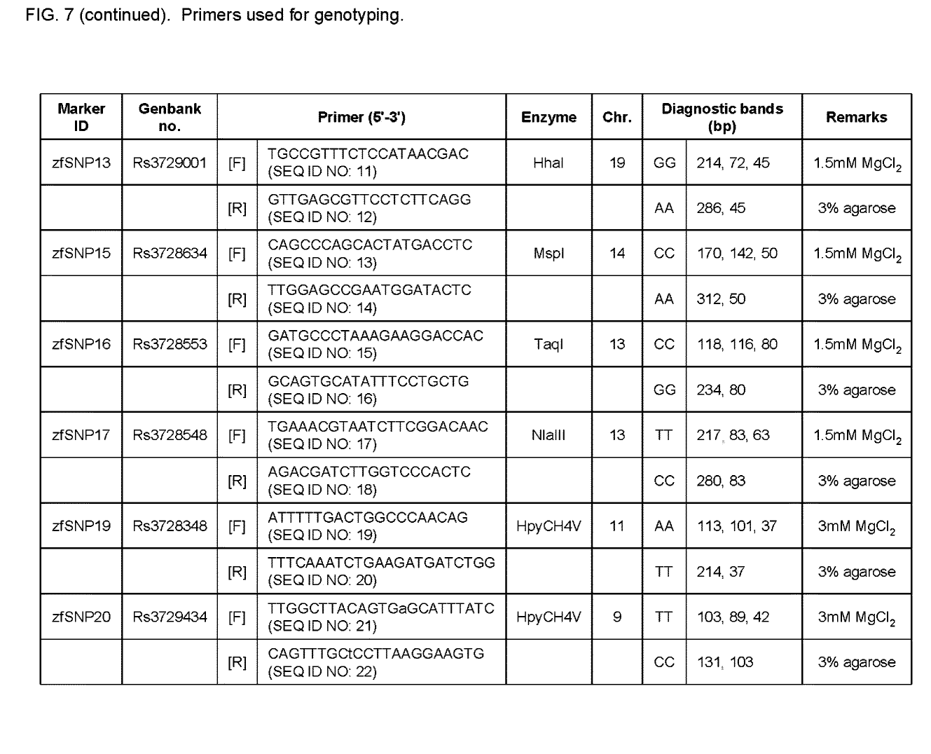

We also developed a DNA fingerprinting analysis using single nucleotide polymorphisms (SNP) to identify the genotype of cloned fish. We selected SNP markers from the SNP database in Genbank based on chromosomal regions and a presence of restriction enzyme cutting site(s) both at the polymorphic nucleotide (diagnostic site) and, if possible, at the adjacent nucleotide (internal control site). We analyzed the genomic region of interest using UCSC genome browser (17) and designed primers using primer3 (18). SNP genotyping was analyzed by restriction fragment length polymorphism (RFLP) following polymerase chain reaction (PCR). Primer sets, PCR condition, restriction enzymes, and diagnostic product sizes used for amplification are shown in FIG. 7, with Chr. indicating the chromosome region of SNP marker and lower cases indicate mutagenic sequence of primers.

Eleven informative SNPs were used to confirm the DNA fingerprints of cloned embryos, donor cells, and donor eggs. Referring now to FIG. 5B, letters indicate genotypes, arrows point to diagnostic bands of each genotype, and IC is an internal control for restriction enzyme activity. The SNP markers were tested and those found to be highly polymorphic among individuals were selected. Table 1 shows genotypes of recipient egg's donor (R), donor cell (D), and cloned embryo (C). For all eleven SNP markers tested, we found a complete matched genetic identity between the donor cell and the cloned fish and no matching with the recipient egg donor was observed.

### Example 3

### Generation of Zebrafish Carrying a Targeted Mutation

A reliable Zebrafish SCNT procedure significantly enhances the usefulness of this model system, e.g., for studies of vertebrate developmental biology and human disease. Donor cells are cultured in vitro and genetically modified by knock-out and knock-in methodologies. These methods of genetic modification can be standard methods similar to or adapted from those previously employed in zebrafish and in other eukaryotic cell culture systems, and can employ targeted (e.g. by sequence homology) or non-targeted integration events, recombinases such as CRE/Lox and FLP/FRT, positive and negative selectable markers, and other standard genetic methodologies. The integration site and disposition of the genetic modification(s) and expression of transgene(s) are optionally confirmed. Wild-type and genetically modified cells are optionally maintained in culture or cryopreserved, providing a stable reservoir of cells which can be used as nuclear donors. Genetically modified cells are then cloned using the methods described in Example 1. Resulting cloned fish are tested to identify individuals carrying the desired genotype(s) or phenotype(s). Healthy and fertile clones can be identified and bred to maintain the genotype, back-crossed to ensure the absence of other undesired genetic modifications, crossed to introduce the genetic modifications into other strains or create combinations of genetic modifications, etc.

Using these methods, the timeline to produce a founder fish carrying the targeted gene is significantly reduced when compared to ES cell chimera technology and other methods, as SCNT offers an unparalleled advantage over others approaches that are currently in use to generate mutants, as it involves no breeding at all, ensuring that the F0 animals are genetically modified. Thus, compared to known methods, the procedure of making germline transgenic fish can be shortened by 6 to 7 months.

TABLE 1

Markers used for genotyping.

SNP identification number (S#)

| | | | | | | | | | | | | |
|---|---|---|---|---|---|---|---|---|---|---|---|---|
| ID | Fish ID | S3 | S5 | S8 | S9 | S10 | S13 | S15 | S16 | S17 | S19 | S20 |

| | | | | | | | | | | | | |
|---|---|---|---|---|---|---|---|---|---|---|---|---|
| R | Fin♀, WT | GG | GA | GC | CC | TT | GA | AA | GG | TC | TA | CC |
| D | Donor, Gol^{-/-} ET 20s | GA | AA | CC | CT | CT | GA | CC | GC | TT | TT | CC |
| C | clone NT2E1, 10dpNT | GA | AA | CC | CT | CT | GA | CC | GC | TT | TT | CC |
| C | clone 9-7 adult fish ♀, 3m | GA | AA | CC | CT | CT | GA | CC | GC | TT | TT | CC |
| R | Fin♀, WT | GG | GA | GC | CC | CT | GG | AC | CC | TT | TT | CT |
| D | Donor, Gol^{-/-} ET 15s | GG | AA | CC | CT | CT | AA | AC | GC | TT | TT | CC |
| C | clone NT5E1, 7dpNT | GG | AA | CC | CT | CT | AA | AC | GC | TT | TT | CC |
| C | clone 9-12 adult fish ♀, 3m | GG | AA | CC | CT | CT | AA | AC | GC | TT | TT | CC |
| R | Fin♀, WT | GG | GG | GG | CC | CT | AA | AA | CC | TT | AA | TT |
| D | Donor, Gol^{-/-} ET 18s | GG | GA | GC | CC | TT | GG | AC | CC | TT | TT | CT |
| C | clone NT8E1, 5dpNT | GG | GA | GC | CC | TT | GG | AC | CC | TT | TT | CT |
| C | clone NT2E1, 6dpNT | GG | GA | GC | CC | TT | GG | AC | CC | TT | TT | CT |
| C | clone NT5E1, 6dpNT | GG | GA | GC | CC | TT | GG | AC | CC | TT | TT | CT |
| R | Fin♀, WT | GG | GA | GG | CT | TT | GA | AA | GC | TT | TA | CT |
| D | Donor, Gol^{-/-} P1♂, AF | GG | GG | GG | CT | CT | GA | CC | CC | TT | TT | TT |
| C | clone NT7E1, 6dpNT | GG | GG | GG | CT | CT | GA | CC | CC | TT | TT | TT |
| R | Fin♀, WT | GG | GA | GG | CC | TT | AA | AC | CC | TT | AA | CC |
| D | Donor, Gol^{-/-} P2♀, AF | GA | AA | CC | CC | TT | GA | CC | GC | TT | TT | CC |
| C | clone NT1E1, 3dpNT | GA | AA | CC | CC | TT | GA | CC | GC | TT | TT | CC |
| R | Fin♀, H2AGFP | GG | AA | GG | CC | CT | GG | AA | GG | TC | TT | CT |
| D | Donor, Gol^{-/-} ET 15s | GA | GA | GC | CT | CC | GA | AC | GC | TT | TT | CT |
| C | clone NT3E1, 14dpNT | GA | GA | GC | CT | CC | GA | AC | GC | TT | TT | CT |
| C | clone NT8E1ab, 7dpNT | GA | GA | GC | CT | CC | GA | AC | GC | TT | TT | CT |
| R | Fin♀ H2AGFP | GG | GG | GG | CC | TT | GA | CA | GG | TT | TT | CC |
| D | Donor, Gol^{-/-} ET 20s | GG | AA | GG | TC | CT | GA | CA | GC | TT | TT | CC |
| C | Clone NT1E1, 4dpNT | GG | AA | GG | TC | CT | GA | CA | GC | TT | TT | CC |
| C | Clone NT2E1, 4dpNT | GG | AA | GG | TC | CT | GA | CA | GC | TT | TT | CC |
| C | Clone NT4E1, 4dpNT | GG | AA | GG | TC | CT | GA | CA | GC | TT | TT | CC |
| R | Fin♀ H2AGFP | GG | AA | GG | CC | CC | GG | AA | GC | TT | TT | TC |
| D | Donor, Gol^{-/-} ET 20s | GG | GA | CG | TT | CT | GA | CC | GC | TT | TT | TC |
| C | Clone NT2E1 | GG | GA | CG | TT | CT | GA | CC | GC | TT | TT | TC |
| C | Clone NT4E1, 10dpNT | GG | GA | CG | TT | CT | GA | CC | GC | TT | TT | TC |
| C | Clone NT5E1 | GG | GA | CG | TT | CT | GA | CC | GC | TT | TT | TC |
| R | Fin♀ H2AGFP | GG | AA | CG | TC | CT | GG | AA | GC | TC | TT | TT |
| D | Donor, Gol^{-/-} ET 20s | GG | AA | CG | TC | CT | AA | CC | GC | TT | TT | TC |
| C | Clone NT2E1, 10dpNT | GG | AA | CG | TC | CT | AA | CC | GC | TT | TT | TC |
| C | Clone NT2E2, 10dpNT | GG | AA | CG | TC | CT | AA | CC | GC | TT | TT | TC |
| C | Clone NT2E3, 12dpNT | GG | AA | CG | TC | CT | AA | CC | GC | TT | TT | TC |
| C | Clone NT3E1, 10dpNT | GG | AA | CG | TC | CT | AA | CC | GC | TT | TT | TC |
| C | Clone NT6E1, 8dpNT | GG | AA | CG | TC | CT | AA | CC | GC | TT | TT | TC |
| C | Clone NT6E2, 8dpNT | GG | AA | CG | TC | CT | AA | CC | GC | TT | TT | TC |
| C | Clone NT6E3, 8dpNT | GG | AA | CG | TC | CT | AA | CC | GC | TT | TT | TC |
| C | Clone NT7E1, 8dpNT | GG | AA | CG | TC | CT | AA | CC | GC | TT | TT | TC |
| R | Fin♀ H2AGFP | GG | GA | CG | CC | CC | GG | CA | GG | TC | TT | CC |
| D | Donor, Gol^{-/-} ET 20s | GG | AA | GG | TT | CC | GA | CC | GC | TT | TT | CC |
| C | Clone NT5E1, 4dpNT | GG | AA | GG | TT | CC | GA | CC | GC | TT | TT | CC |
| C | Clone NT7E2, 4dpNT | GG | AA | GG | TT | CC | GA | CC | GC | TT | TT | CC |
| R | Fin♀ H2AGFP | GG | GA | CG | CC | CC | GG | AA | GC | TT | TT | TT |
| D | Donor, Gol^{-/-} ♂ AF | GG | AA | GG | TC | CT | GA | CA | GG | TT | TT | CC |
| C | Clone NT5E1, 1dpNT | GG | AA | GG | TC | CT | GA | CA | GG | TT | TT | CC |
| R | Fin♀ H2AGFP | GG | GA | CG | TT | CT | GG | CC | GG | TC | TT | TC |
| D | Donor, Gol^{-/-} ♂AF | GG | AA | GG | TC | CT | GA | CC | GG | TT | TT | CC |
| C | Clone NT5E1, 2dpNT | GG | AA | GG | TC | CT | GA | CC | GG | TT | TT | CC |
| R | Fin♀ H2AGFP - HG1 | GA | AA | GG | TC | CC | GA | AA | GG | TT | TT | TC |
| D | Donor, Gol^{-/-} ♂AF | GA | AA | GG | TC | CC | GA | CC | CC | TT | TT | CC |
| C | Clone NT3E1, 2dpNT | GA | AA | GG | TC | CC | GA | CC | CC | TT | TT | CC |
| C | Clone NT3E2, 2dpNT | GA | AA | GG | TC | CC | GA | CC | CC | TT | TT | CC |
| R | Fin♀ H2AGFP | GG | AA | CG | CC | CC | GG | CA | CC | TC | TT | TC |
| D | Donor, Gol^{-/-} ♂AF | GA | AA | GG | TC | CC | GA | CC | CC | TT | TT | CC |
| C | Clone NT2E1, 1dpNT | GA | AA | GG | TC | CC | GA | CC | CC | TT | TT | CC |
| R | Fin♀ TAB2 | GA | AA | CG | CC | TT | GA | CA | GC | TT | TT | TC |
| D | Donor, HGn62A-Leo ET 15s | GA | GA | GG | CC | CT | GG | CC | GG | TT | TT | CC |
| C | Clone NT1, 1 month | GA | GA | GG | CC | CT | GG | CC | GG | TT | TT | CC |
| C | Clone NT2, 1 month | GA | GA | GG | CC | CT | GG | CC | GG | TT | TT | CC |
| C | Clone NT3, 1 month | GA | GA | GG | CC | CT | GG | CC | GG | TT | TT | CC |
| C | Clone NT1E1, 4dpNT | GA | GA | GG | CC | CT | GG | CC | GG | TT | TT | CC |
| C | Clone NT1E2, 19dpNT | GA | GA | GG | CC | CT | GG | CC | GG | TT | TT | CC |
| C | Clone NT3E1, 10dpNT | GA | GA | GG | CC | CT | GG | CC | GG | TT | TT | CC |
| C | Clone NT3E2, 4dpNT | GA | GA | GG | CC | CT | GG | CC | GG | TT | TT | CC |
| C | Clone NT3E3, 4dpNT | GA | GA | GG | CC | CT | GG | CC | GG | TT | TT | CC |
| C | Clone NT3E4, 4dpNT | GA | GA | GG | CC | CT | GG | CC | GG | TT | TT | CC |
| C | Clone NT4F1, 4dpNT | GA | GA | GG | CC | CT | GG | CC | GG | TT | TT | CC |
| R | Fin♀ TAB2 | GA | AA | CG | CC | TT | GA | CA | GC | TT | TT | TC |
| D | Donor, HGn62A ET 18s | GG | GA | GG | CC | CT | GA | CC | GG | TT | TT | CC |
| C | Clone NT1, 1 month | GG | GA | GG | CC | CT | GA | CC | GG | TT | TT | CC |
| C | Clone NT2, 1 month | GG | GA | GG | CC | CT | GA | CC | GG | TT | TT | CC |
| C | Clone NT1E2, 16dpNT | GG | GA | GG | CC | CT | GA | CC | GG | TT | TT | CC |
| C | Clone NT2E3, 3dpNT | GG | GA | GG | CC | CT | GA | CC | GG | TT | TT | CC |
| R | Fin♀ TAB2 | GA | AA | CG | CC | TT | GA | CA | GC | TT | TT | TC |
| D | Donor HGn8E ET 18s | GG | GG | GG | CC | CT | GG | AA | GG | TT | TT | CC |
| C | Clone NT2E1, 14dpNT | GG | GG | GG | CC | CT | GG | AA | GG | TT | TT | CC |
| C | Clone NT1E1, 4dpNT | GG | GG | GG | CC | CT | GG | AA | GG | TT | TT | CC |
| R | Fin♀ TAB2 | GA | AA | CG | CC | TT | GG | CA | GC | TT | TT | TC |
| D | Donor ♂HGn8E leo AF | GG | GG | GG | CC | CT | GA | AA | GG | TT | TT | CC |
| C | Clone NT1E1, 4dpNT | GG | GG | GG | CC | CT | GA | AA | GG | TT | TT | CC |
| C | Clone NT2E1, 4dpNT | GG | GG | GG | CC | CT | GA | AA | GG | TT | TT | CC |
| R | Fin♀ TAB2 | GA | AA | CG | CC | TT | GA | CA | GC | TT | TT | TC |
| D | Donor, ♂HGn28A leo AF | GG | GG | GG | CC | CT | GA | AA | GG | TT | TT | CC |
| C | Clone NT4E1, 4dpNT | GG | GG | GG | CC | CT | GA | AA | GG | TT | TT | CC |
| C | Clone NT3E1, 4dpNT | GG | GG | GG | CC | CT | GG | AA | GG | TT | TT | CC |
| R | Fin♀ TAB2 | GA | AA | CG | CC | TT | GG | CA | GC | TT | TT | TC |
| D | Donor, ♀HGn62A leo AF | GA | GA | GG | CC | CT | GG | CC | GG | TT | TT | CC |
| C | Clone NT4E1, 7dpNT | GA | GA | GG | CC | CT | GG | CC | GG | TT | TT | CC |
| R | Fin♀ TAB2 | GA | AA | CG | CC | TT | GA | CA | GC | TT | TT | TC |
| D | Donor, ♂HGn8E leo AF | GG | GG | GG | CC | CT | GA | AA | GG | TT | TT | CC |
| C | Clone NT2E1, 2dpNT | GG | GG | GG | CC | CT | GA | AA | GG | TT | TT | CC |
| C | Clone NT5E1, 2dpNT | GG | GG | GG | CC | CT | GA | AA | GG | TT | TT | CC |

While the invention has been described by way of examples and preferred embodiments, it is understood that the words which have been used herein are words of description, rather than words of limitation. Changes may be made, within the purview of the appended claims, without departing from the scope and spirit of the invention in its broader aspects. Although the invention has been described herein with reference to particular means, materials, and embodiments, it is understood that the invention is not limited to the particulars disclosed. The invention extends to all equivalent structures, means, and uses which are within the scope of the appended claims.

Each document cited herein is hereby incorporated by reference in its entirety, as are the references cited in those documents.

### REFERENCES

The citations in the disclosure above refer to the numbered references below.
1. Nüsslein-Volhard, C. & Dahm, R. Zebrafish: A practical approach, 1st ed. (Oxford University Press, Oxford, 2002). 100 (1999).
3. Lee, K. Y., Huang, H., Ju, B., Yang, Z. & Lin, S. Cloned zebrafish by nuclear transfer from long-term-cultured cells. Nat Biotechnol 20, 795-799 (2002).
4. Amsterdam, A. et al. Identification of 315 genes essential for early zebrafish development. Proc Natl Acad Sci USA 101, 12792-12797 (2004).
5. Talbot, W. S. & Hopkins, N. Zebrafish mutations and functional analysis of the vertebrate genome. Genes Dev 14, 755-762 (2000).
6. Anderson, K. V. & Ingham, P. W. The transformation of the model organism: A decade of developmental genetics. Nat Genet. 33, 285-293 (2003).
7. Fan, L., Moon, J., Crodian, J. & Collodi, P. Homologous recombination in zebrafish es cells. Transgenic Res 15, 21-30 (2006).
8. Ma, C., Fan, L., Ganassin, R., Bols, N. & Collodi, P. Production of zebrafish germ-line chimeras from embryo cell cultures. Proc Natl Acad Sci USA 98, 2461-2466 (2001).
9. Wilmut, I., Schnieke, A. E., McWhir, J., Kind, A. J. & Campbell, K. H. S. Viable offspring derived from fetal and adult mammalian cells. Nature 385, 810-813 (1997).
10. Amores, A. & Postlethwait, J. H. Banded chromosomes and the zebrafish karyotype. Methods Cell Biol 60, 323-338 (1999).
11. Lamason, R. L. et al. Slc24a5, a putative cation exchanger, affects pigmentation in zebrafish and humans. Science 310, 1782-1786 (2005).
12. Nagayoshi, S. et al. Insertional mutagenesis by the tol2 transposon-mediated enhancer trap approach generated mutations in two developmental genes: Tcf7 and synembryn-like. Development 135, 159-169 (2008).
13. Pauls, S., Geldmacher-Voss, B. & Campos-Ortega, J. A. A zebrafish histone variant h2a.F/z and a transgenic h2a.F/z:Gfp fusion protein for in vivo studies of embryonic development. Dev Genes Evol 211, 603-610 (2001).
14. Egli, D., Rosains, J., Birkhoff, G. & Eggan, K. Developmental reprogramming after chromosome transfer into mitotic mouse zygotes. Nature 447, 679-685 (2007).
15. Siripattarapravat, K., Busta, A., Steibel, J. P. & Cibelli, J. Characterization and in vitro control of mpf activity in zebrafish eggs. Zebrafish (2009).
16. Corley-Smith, G. E., Brandhorst, B. P., Walker, C. & Postlethwait, J. H. Production of haploid and diploid androgenetic zebrafish (including methodology for delayed in vitro fertilization). Methods Cell Biol 59, 45-60 (1999).
17. Kent, W. J. Blat—the blast-like alignment tool. Genome Res 12, 656-664 (2002).
18. Rozen, S. & Skaletsky, H. Primer3 on the www for general users and for biologist programmers. Methods Mol Biol 132, 365-386 (2000).
19. Westerfield, M. The zebrafish book, 4 ed. (University of Oregon Press, Eugene, 2000).
20. Lin, T. M., Tsai, J. L., Lin, S. D., Lai, C. S. & Chang, C. C. Accelerated growth and prolonged lifespan of adipose tissue-derived human mesenchymal stem cells in a medium using reduced calcium and antioxidants. Stem Cells Dev 14, 92-102 (2005).
21. U.S. Pat. No. 7,332,647.
22. Huang H. et al., Cloning Stem Cells. 2003; 5(4):333-7.

1. A method for making an enucleated cell comprising:
visualizing the nucleus of a cell; and
irradiating the nucleus of the cell with a radiation source;
whereby the nuclear DNA or nucleus of the cell is ablated.

2. The method of claim 1, wherein the cell is an oocyte or primordial germ cell.

3. The method of claim 1, wherein the enucleated cell is of fish origin.

4. The method of claim 3 wherein the fish is selected from the group consisting of zebrafish, koi, medaka fish, Boraras, Cyprinus, Danio, Devario, Pseudorasbora, Rasbora, Tanichthys, Trigonostigma, a hybrid between two strains of a species in one of the foregoing genera, a hybrid between two species within one of the foregoing genera, and a hybrid between two species of two different foregoing genera.

5. The method of claim 1 wherein the enucleated cell is a fish oocyte that would undergo parthenogenetic activation upon chorion removal.

6. The method of claim 1 wherein the radiation is a source of electromagnetic radiation selected from the group consisting of gamma rays, X-rays, ultraviolet, visible light, infrared light, terahertz radiation, microwaves, and radio waves.

7. The method of claim 1 wherein the radiation source is selected from the group consisting of a laser, an LED, a xenon arc lamp, a deuterium arc lamp, a halogen lamp, a mercury-xenon arc lamp, a metal-halide arc lamp, a tungsten-halogen incandescent lamp, an incandescent lamp, a fluorescent lamp, a high-intensity discharge lamp, a gas discharge lamp, and an electric arc.

8. The method of claim 1, wherein visualizing the cell nucleus comprises contacting the cell with a nuclear stain.

9. The method of claim 8, wherein the nuclear stain is selected from the group consisting of Hoechst 33258, Hoechst 33342, 4′,6-diamidino-2-phenylindole (DAPI), Acridine orange, Nile blue, Safranin, SYBRgreen, SYBR Green II, SYBR Gold, Oxazole Yellow, Thiazole Orange, PicoGreen, and any combination thereof.

10. The method of claim 1, wherein visualizing the cell nucleus comprises observing expression of a fluorescent protein in the cell nucleus, wherein the fluorescent protein comprises a green, blue, yellow, or cyan fluorescent protein.

11. The method of claim 10, wherein the fluorescent protein is a fusion protein comprising a histone or a nuclear localization sequence.

12. A method of producing a nuclear transplant cell comprising:
providing an enucleated cell by method of claim 1, and introducing donor cell-derived genetic material into the enucleated cell.

13. The method of claim 12 wherein introducing donor genetic material into the enucleated cell is performed by a method selected from the group consisting of:
transplanting a donor cell-derived nucleus into the enucleated cell;
transplanting donor cell-derived chromosomal DNA into the enucleated cell;
fusing a donor cell with all or part of said enucleated cell; and
transplanting a donor cell into said enucleated cell.

14. The method of claim 12, wherein the enucleated cell is an oocyte and donor genetic material is introduced through the micropyle of the enucleated oocyte.

15. The method of claim 12, wherein the donor cell is transgenic.

16. The method of claim 12, wherein the donor cell is of the same species as the enucleated cell, is of the same genus as the enucleated cell, is of a different species than the enucleated cell, or is of a different genus than the enucleated cell.

17. The method of claim 12, wherein the donor nucleus is mammalian, human, fish, zebrafish, koi, medaka fish, Boraras, Cyprinus, Danio, Devario, Pseudorasbora, Rasbora, Tanichthys, Trigonostigma, a hybrid between two strains of a species in one of the foregoing genera, a hybrid between two species within one of the foregoing genera, and a hybrid between two species of two different foregoing genera.

18. An enucleated cell, produced by the method of claim 1.

19. A nuclear transplant cell, produced by the method of claim 12.

20. A non-human organism produced from the nuclear transplant cell of claim 19.

21. The organism of claim 20 which is selected from the group consisting of: fish, zebrafish, koi, medaka fish, Boraras, Cyprinus, Danio, Devario, Pseudorasbora, Rasbora, Tanichthys, Trigonostigma, a hybrid between two strains of a species in one of the foregoing genera, a hybrid between two species within one of the foregoing genera, and a hybrid between two species of two different foregoing genera.

22. The method of claim 1 which is a high-throughput method.


SEQUENCE LISTING (22 sequences)

SEQ ID NO: 1 - DNA, 21 bp, Danio rerio
tgcaactgaccgatgtatttg

SEQ ID NO: 2 - DNA, 20 bp, Danio rerio
cctttcctccctctgatgtg

SEQ ID NO: 3 - DNA, 20 bp, Danio rerio
ttggctggcttgagattgat

SEQ ID NO: 4 - DNA, 27 bp, Danio rerio
tcatcagagtcagtgaataatttttgt

SEQ ID NO: 5 - DNA, 22 bp, Danio rerio
gaccgtctagaggtttctgtgg

SEQ ID NO: 6 - DNA, 19 bp, Danio rerio
cattctccacgaccactgc

SEQ ID NO: 7 - DNA, 20 bp, Danio rerio
tgcgcttttacagactgtgc

SEQ ID NO: 8 - DNA, 20 bp, Danio rerio
agcaatgcttcactccatcc

SEQ ID NO: 9 - DNA, 20 bp, Danio rerio
aatcctggttggcttgtagc

SEQ ID NO: 10 - DNA, 21 bp, Danio rerio
agcagtacttttagcccttgg

SEQ ID NO: 11 - DNA, 20 bp, Danio rerio
tgccgtttctccataacgac

SEQ ID NO: 12 - DNA, 20 bp, Danio rerio
gttgagcgttcctcttcagg

SEQ ID NO: 13 - DNA, 20 bp, Danio rerio
cagcccagcactatgacctc

SEQ ID NO: 14 - DNA, 20 bp, Danio rerio
ttggagccgaatggatactc

SEQ ID NO: 15 - DNA, 21 bp, Danio rerio
gatgccctaaagaaggaccac

SEQ ID NO: 16 - DNA, 20 bp, Danio rerio
gcagtgcatatttcctgctg

SEQ ID NO: 17 - DNA, 22 bp, Danio rerio
tgaaacgtaatcttcggacaac

SEQ ID NO: 18 - DNA, 20 bp, Danio rerio
agacgatcttggtcccactc

SEQ ID NO: 19 - DNA, 20 bp, Danio rerio
atttttgactggcccaacag

SEQ ID NO: 20 - DNA, 23 bp, Danio rerio
tttcaaatctgaagatgatctgg

SEQ ID NO: 21 - DNA, 23 bp, Danio rerio
ttggcttacagtgagcatttatc

SEQ ID NO: 22 - DNA, 22 bp, Danio rerio
cagtttgctccttaaggaagtg